(19) United States
(12) Patent Application Publication
(10) Pub. No.: US 2024/0261539 A1
(43) Pub. Date: Aug. 8, 2024

(54) HYPOTUBE WITH PROGRESSIVE BENDING STIFFNESS AND IMPROVED TENSILE STRENGTH

(71) Applicants: STRYKER CORPORATION, Kalamazoo, MI (US); STRYKER EUROPEAN OPERATIONS LIMITED, Carrigtwohill, Co. Cork (IE)

(72) Inventors: Clay William Northrop, Salt Lake City, UT (US); Murtagh M. Murphy, Jacob's Island, Cork (IE)

(73) Assignees: STRYKER CORPORATION, Kalamazoo, MI (US); STRYKER EUROPEAN OPERATIONS LIMITED, Carrigtwohill, Co. Cork (IE)

(21) Appl. No.: 18/640,302

(22) Filed: Apr. 19, 2024

Related U.S. Application Data
(63) Continuation of application No. PCT/US2023/069963, filed on Jul. 11, 2023.
(60) Provisional application No. 63/400,571, filed on Aug. 24, 2022.

(51) Int. Cl. A61M 25/00; A61M 25/09

(52) U.S. Cl. CPC A61M 25/0054; A61M 25/09; A61M 2025/09091; A61M 2025/09133; A61M 2025/09166; A61M 2025/09175; A61M 2205/0216; A61M 2205/0222; A61M 2205/0238; A61M 2205/025; A61M 2205/0266; A61M 2205/32

(57) ABSTRACT

A tubular support structure for use in an elongate intravascular medical device comprises an elongate tubular body, a patterned frame structure formed within the elongate tubular body, an inner lumen axially disposed within the elongate tubular body, and a first set of floating tabs axially spaced apart along the patterned frame structure. Each of the first set of floating tabs has a cantilevered end affixed to the patterned frame structure and a free end configured for translating relative to the patterned frame structure and then engaging the patterned frame structure as the tubular support structure is laterally deflected in a first bending direction.

### RELATED APPLICATION DATA

This application is a continuation of International Patent Application No. PCT/US2023/069963, filed on Jul. 11, 2023, which claims priority to U.S. Provisional Patent Application No. 63/400,571, filed Aug. 24, 2022, the disclosures of all of which are hereby incorporated herein by reference in their entirety into the present application.

### FIELD

The present disclosure relates generally to medical devices, and, more particularly, to support structures for intravascular medical devices.

### BACKGROUND

The use of intravascular medical devices for accessing and treating various types of diseases, such as vascular defects, is well-known. For example, a suitable intravascular catheter may be inserted into the vascular system of a patient. A commonly used vascular application to access a target site in a patient involves inserting a guidewire through an incision in the femoral artery near the groin, and advancing the guidewire until it reaches the target site. Then, an intravascular catheter is advanced over the guidewire via a lumen in the intravascular catheter until an open distal end of the intravascular catheter is disposed at the target site. Alternatively, the intravascular catheter may be introduced into the patient after the guidewire had been withdrawn, leaving a guide sheath for the intravascular catheter to navigate through the vasculature of the patient within the guide sheath. In the case of treating vascular defects, simultaneously or after placement of the distal end of the intravascular catheter at the target site, an intravascular implant may be advanced through the lumen of the catheter via a delivery wire.

In certain applications, such as neurovascular treatment, intravascular medical devices, such as guidewires, catheters, guide sheaths, and intravascular implant delivery wires, are required to navigate tortuous and intricate vasculature, including travel within relatively fragile blood vessels in the brain, and are often required to change direction and to even double back on themselves. Thus, such intravascular medical devices should have suitable trackability, flexibility, pushability (axial rigidity), torqueability (rotation), kink resistance, and pullability (tensile strength) to successfully navigate the vasculature of a patient, such as cerebral, coronary, and peripheral vasculature. Trackability is the measure of the ability of a intravascular medical device to navigate through the vasculature of a patient. Flexibility is the measure of lateral bending stiffness along the length of an intravascular medical device and is a significant contributor to the trackability of the elongate intravascular medical device. Pushability is the measure of transmission of proximally applied axial forces to the distal end of an intravascular medical device. Torqueability is the measure of the transmission of proximally applied rotational forces to the distal end of an intravascular medical device and aids navigation through tortuous paths in the vasculature of the patient. Kink resistance is the measure of the ability of an intravascular medical device to maintain its cross-sectional profile, especially the inner lumen (if it exists), when bent around a radius. Pullability is the measure of the ability of an intravascular medical device to transmit axial tensile forces without plastically deforming or breaking the elongate intravascular medical device.

Suitable flexibility and kink resistance of these intravascular medical devices allow them to navigate through moderate bends in the vasculature with relatively little tracking force (due to lower lateral forces) and also relatively tight bends without breaking, permanently deforming, and prolapsing (due to adequate support and resistance). Furthermore, it may not only be desirable to push these intravascular medical devices through the vasculature, it may be desirable to pull these intravascular medical devices within the vasculature, e.g., if such intravascular medical devices get stuck (e.g., if prolapsed within the vasculature of the patient, become trapped by vasospasm or interference with another intravascular medical device, etc.) or are located in the wrong or undesired vessel within the vasculature. Thus, it important that such intravascular medical devices have the necessary axial tensile strength to avoid damaging or otherwise plastically deforming them. Any intravascular medical device must meet a minimum tensile strength in order to be safe for use. In many instances, secondary elements must be added to the elongate intravascular medical device in order simply to meet this tensile strength, or existing elements must be made more robust (larger or of stronger materials) than would be otherwise necessary, which typically makes them stiffer than would be otherwise necessary. Additionally, the space taken up by the secondary strengthening elements or the more robust existing elements becomes unavailable for other elements that could improve device performance. Further, these added tensile elements or more robust elements may increase the lateral bending stiffness of the device to a degree that has a negative impact on other performance characteristics, such as trackability. Thus, if it is possible to gain improved tensile strength through performance-specific design of elements that are already present within the elongate intravascular medical device, performance advantage can be gained. Thus, by using an appropriately sized intravascular medical device having the requisite performance characteristics, such as pushability, torqueability, pullability, and distal tip flexibility, virtually any target site in the vascular system may be accessed, including those within the tortuous cerebral and peripheral vasculature. However, achieving a balance between these performance characteristics can be difficult.

Presently, there are numerous microcatheter and guidewire designs with hypotubes that attempt to achieve this balance. In general, a hypotube is a long thin-walled tube formed from a metal or a metal alloy, such as stainless steel, nickel titanium alloy (e.g., nitinol), or the like. A hypotube often has micro-engineered features along its length. The distal end of a hypotube may have a slotted pattern that enhances its flexibility, while providing sufficient axial rigidity to maintain the pushability of the hypotube through the vasculature of a patient. In some instances, a polymer jacket may be applied to the outer diameter of the slotted hypotube to provide a seal and to also minimize any exterior surface roughness imparted by the slots of the hypotube while still providing flexibility. This outer jacket may fill the apertures/slots in the hypotube, and even coat the internal surface of the hypotube. For applications, such as applications of neurovascular treatment, that involve passing various other devices, agents, and/or fluids into a body lumen or cavity in a patient by the catheter, the properties of the inner surface of the lumen(s) of the catheter may significantly impact the performance of the catheter. In particular, the lubricity of the inner surface may affect the ability to pass other devices, agents, and/or fluids through the lumen(s) of the catheter. To enhance lubricity, a low friction inner polymer liner (e.g., polytetrafluoroethylene (PTFE)) can surround the lumen of a catheter. The inner polymer liner may provide a lubricious inner surface to facilitate passing guidewires, pacing leads, or other devices through the lumen of the catheter.

While these slotted hypotube designs may generally strike a fair balance between pushability, pullability, torqueability, kink resistance, and distal tip flexibility, such slotted hypotube designs may still be improved. For example, slotted hypotube designs generally have a relatively low isotropic bending stiffness (i.e., the same bending stiffness in all radial directions), such that the elongate medical devices may be easily advanced through low to moderate bends in the vasculature with low tracking force. However, such intravascular medical devices are still prone to prolapse when being introduced through high bends in the vasculature. Furthermore, the slotted pattern of the hypotubes typically results in ribs or struts that are transverse or orthogonal to the longitudinal axes of the hypotubes. Thus, in response to a tensile force applied to such slotted hypotubes (e.g., if the elongate medical device is being pulled), lateral bending forces are applied to the ribs or struts of the supporting structure. These lateral forces typically create high bending moments, and thus high localized stress in the tubular structure. As such, the ribs or struts are prone to more easily deform in the presence of tensile forces applied to the slotted hypotubes, thereby plastically deforming or breaking the elongated medical devices at relatively low tensile forces. Additionally, if an intravascular medical device is prone to stretching under tension, whether elastically or plastically, this reduces the precision with which it can be navigated within the vasculature, decreasing its performance. Thus, slotted hypotubes may need to be composed of a relatively strong, but relatively stiff, material (e.g., a metal such as stainless steel) rather than a more flexible, but less strong material (e.g., a metal alloy such as nickel titanium (nitinol)). Thus, the tensile strength and stiffness of such slotted hypotubes typically must be sacrificed to provide sufficient bending flexibility to the slotted hypotubes.

There, thus, is a need for slotted hypotube designs that overcome the foregoing challenges.

### SUMMARY

The present inventions comprise a tubular support structure for use in an elongate intravascular medical device. The tubular support structure comprises an elongate tubular body (e.g., a hypotube), a patterned frame structure formed within the elongate tubular body, and an inner lumen axially disposed within the elongate tubular body. In one embodiment, the patterned frame structure has a plurality of substantially transverse slots disposed at least partially within the elongate tubular body, and the substantially transverse slots are axially spaced apart along the tubular support structure, thereby forming a plurality of substantially transverse members and a plurality of connecting members rigidly coupling the substantially transverse members together. The connecting members may axially extend along the patterned frame structure, and the substantially transverse slots may be disposed entirely through the elongate tubular body. One embodiment of an elongate intravascular medical device may comprise an elongate polymer tube and the tubular support structure coaxially disposed within the polymer tube. In this embodiment, the tubular support structure may be disposed at a distal end of the polymer tube or proximal to the distal end of the polymer tube. Another embodiment of an elongate intravascular medical device may comprise a core wire and the tubular support structure disposed over a distal end of the core wire. Still another embodiment of an elongate intravascular medical device may comprise the tubular support structure, and an inner polymer liner disposed within the inner lumen of the tubular body of the tubular support structure.

In accordance with a first aspect of the present inventions, the tubular support structure comprises a first set of floating tabs axially spaced apart along the patterned frame structure. Each of the first set of floating tabs has a cantilevered end affixed to the patterned frame structure (e.g., a substantially transverse member of the patterned frame structure) and a free end configured for translating relative to the patterned frame structure and then engaging the patterned frame structure as the tubular support structure is laterally deflected in a first bending direction. The first set of floating tabs may, e.g., be circumferentially aligned on the patterned frame structure or circumferentially offset on the patterned frame structure.

In one embodiment, each of the first set of floating tabs is configured for translating relative to the patterned frame structure when the tubular support structure is in a primary lateral deflection range, and engaging the patterned frame structure when the tubular support structure is in a secondary lateral deflection range greater than the primary lateral deflection range. In this embodiment, the tubular support structure may have a primary bending stiffness (e.g., greater than 0.00001 in²-lb) when the tubular support structure is in the primary lateral deflection range, and one or more secondary bending stiffnesses greater than the primary bending stiffness when the tubular support structure is in the secondary lateral deflection range. The highest of the secondary bending stiffness(es) may be less than five times, and preferably less than two times, the primary bending stiffness. In this embodiment, the primary bending stiffness may be radially isotropic, while each of the secondary bending stiffness(es) may be radially anisotropic, such that each secondary bending stiffness has at least one relatively low magnitude circumferential region and at least one relatively high magnitude circumferential region. The magnitude of each of the relatively low magnitude circumferential region(s) may be equal to or higher than the magnitude of the primary bending stiffness.

In another embodiment, the free end of each of the first set of floating tabs is configured for translating relative to the patterned frame structure and then engaging the patterned frame structure as the tubular support structure is axially stretched. In this embodiment, each of the first set of floating tabs may be configured for translating relative to the patterned frame structure when the tubular support structure is in a first axial stretch range, and engaging the patterned frame structure when the tubular support structure is in a second axial stretch range greater than the first axial stretch range. The tubular support structure may have a primary tensile stiffness when the tubular support structure is in the first axial stretch range, and one or more secondary tensile stiffnesses greater than the first tensile stiffness when the tubular support structure is in the second axial stretch range.

In still another embodiment, the first set of floating tabs is configured for incrementally engaging the patterned frame structure as the tubular support structure is lateral deflected in the first bending direction. For example, at least two of the floating tabs may have different lengths.

In yet another embodiment, the tubular support structure further comprises a second set of floating tabs axially spaced apart along the patterned frame structure and circumferentially offset from the first set of floating tabs. Each of the second set of floating tabs may have a cantilevered end affixed to the patterned frame structure (e.g., a substantially transverse member of the patterned frame structure) and a free end configured for translating relative to the patterned frame structure and then engaging the patterned frame structure as the tubular support structure is laterally deflected in a second bending direction different from the first bending direction. In this embodiment, the second set of floating tabs may be circumferentially offset from the first set of floating tabs by one hundred eighty degrees, and the second bending direction may be opposite the first bending direction. In this embodiment, the first set of floating tabs may be configured for translating relative to the patterned frame structure in a first axial direction when the tubular support structure is laterally deflected in the first direction, and the second set of floating tabs may be configured for translating relative to the frame in a second axial direction opposite the first axial direction. For example, the first set of floating tabs may be configured for continuing to translate in the first axial direction relative to the patterned frame structure after all of the second set of floating tabs have engaged the patterned frame structure, and the second set of floating tabs may be configured for continuing to translate in the second axial direction relative to the patterned frame structure after all of the first set of floating tabs have engaged the patterned frame structure.

In yet another embodiment, each of the first set of floating tabs comprises a stem element and an enlarged element that respectively form the cantilevered end and free end of the respective tab. For example, each of the first set of floating tabs may be T-shaped. In this embodiment, the patterned frame structure may comprise a plurality of retainer openings disposed at least partially within the elongate tubular body, and the enlarged element of each of the first set of floating tabs may be configured for translating within a respective one of the retainer openings and then engaging an abutment edge of the respective retainer opening as the tubular support structure is laterally deflected in a first bending direction. Each of the retainer openings may be coextensive with a respective one of the substantially transverse slots, in which case, the stem element of each of the first set of floating tabs may extend from the respective transverse member, across a respective one of the substantially transverse slots, and into the respective retainer opening. Each adjacent pair of substantially transverse members may comprise a pair of extensions that form a channel between a respective one of the retainer openings and a respective one of the substantially transverse slots that are coextensive with each other, in which case, the stem element of each of the first set of floating tabs may reside within a respective one of the channels, and each pair of extensions may define the abutment edge of the respective retainer opening. Each pair of extensions may be configured for laterally flexing when the enlarged element of the respective floating tab engages the abutment edge of the respective retainer opening.

In accordance with a second aspect of the present inventions, the tubular support structure comprises a first set of mechanical property modulating elements axially spaced apart along the patterned frame structure. The first set of mechanical property modulating elements is configured for incrementally increasing a finite bending stiffness (e.g., greater than 0.00001 in²-lb) of the tubular support structure in response to laterally deflecting the tubular support structure in a first bending direction. The infinite bending stiffness of the tubular support structure may, e.g., be increased by less than 500%, and preferably less than 200%. The first set of mechanical property modulating elements may, e.g., be circumferentially aligned on the patterned frame structure or circumferentially offset on the patterned frame structure.

In one embodiment, each of the first set of mechanical property modulating elements comprises a floating tab having a cantilevered end affixed to the patterned frame structure and a free end configured for translating relative to the patterned frame structure and then engaging the patterned frame structure as the tubular support structure is laterally deflected in the first bending direction, thereby increasing the finite bending stiffness of the tubular support structure.

In another embodiment, the first set of mechanical property modulating elements is further configured for increasing a finite tensile stiffness of the tubular support structure in response to axially stretching the tubular support structure. In still another embodiment, the first set of mechanical property modulating elements is configured for incrementally increasing the finite bending stiffness of the tubular support structure multiple times in response to laterally deflecting the tubular support structure in the first bending direction.

In still another embodiment, the finite bending stiffness of the patterned tubular support structure is increased from an initial radially isotropic primary bending stiffness to a radially anisotropic secondary bending stiffness, such that the secondary bending stiffness has at least one relatively low magnitude circumferential region and at least one relatively high magnitude circumferential region. In this embodiment, the magnitude of each of the relatively low magnitude circumferential region(s) may be equal to the magnitude of the primary bending stiffness or higher than the magnitude of the primary bending stiffness.

In yet another embodiment, the tubular support structure further comprises a second set of mechanical property modulating elements axially spaced apart along the patterned frame structure and circumferentially offset from the first set of mechanical property modulating elements. The second set of mechanical property modulating elements is configured for incrementally increasing a secondary bending stiffness of the tubular support structure in response to laterally deflecting the tubular support structure in a second bending direction different from the first bending direction. In this embodiment, the second set of mechanical property modulating elements is circumferentially offset from the first set of mechanical property modulating elements by one hundred eighty degrees, and the second bending direction is opposite the first bending direction. In this case, the second set of mechanical property modulating elements may not contribute to the increase in the primary bending stiffness of the tubular support structure when the tubular support structure is laterally deflected in the first bending direction, and the first set of mechanical property modulating elements may not contribute to the increase in the secondary bending stiffness of the tubular support structure when the tubular support structure is laterally deflected in the second bending direction.

In accordance with a third aspect of the present inventions, the tubular support structure comprises a first set of mechanical property modulating elements circumferentially spaced apart around the patterned frame structure. The first set of mechanical property modulating elements is configured for incrementally increasing a finite tensile stiffness (e.g., greater than 0.05 lbs) of the tubular support structure in response to axially stretching the tubular support structure. The finite tensile stiffness of the tubular support structure may, e.g., be increased by more than 50%, and preferably, more than 100%. The first set of mechanical property modulating elements may, e.g., be circumferentially aligned on the patterned frame structure.

In one embodiment, each of the first set of mechanical property modulating elements comprises a floating tab having a cantilevered end affixed to the patterned frame structure and a free end configured for translating relative to the patterned frame structure and then engaging the patterned frame structure as the tubular support structure is axially stretched, thereby increasing the finite tensile stiffness of the tubular support structure.

In another embodiment, the tubular support structure further comprises a second set of mechanical property modulating elements circumferentially spaced apart around the patterned frame structure and axially spaced apart from the first set of mechanical property modulating elements. The second set of mechanical property modulating elements is configured for further increasing the finite tensile stiffness of the tubular support structure in response to axially stretching the tubular support structure.

In still another embodiment, the first and second sets of mechanical property modulating elements are configured for incrementally increasing the finite tensile stiffness of the tubular support structure multiple times in response to axially stretching the tubular support structure.

In yet another embodiment, the tubular support structure has a finite bending stiffness that is incrementally increased as the finite tensile stiffness is incrementally increased. For example, the first set of mechanical property modulating elements may be configured for incrementally increasing the finite bending stiffness as the finite tensile stiffness is incrementally increased.

In accordance with a fourth aspect of the present inventions, the tubular support structure comprises a plurality of mechanical property modulating elements disposed on the patterned frame structure (e.g., affixed to substantially transverse members of the patterned frame structure). The plurality of mechanical property modulating elements is configured for modulating a radially isotropic bending stiffness of the tubular support structure in response to laterally deflecting the patterned frame structure in one or more bending directions, such that the tubular support structure has a radially anisotropic bending stiffness.

In one embodiment, each of the plurality of mechanical property modulating elements comprises a floating tab having a cantilevered end affixed to the patterned frame structure and a free end configured for translating relative to the patterned frame structure and then engaging the patterned frame structure as the tubular support structure is laterally deflected in the first bending direction, thereby modulating the radially isotropic bending stiffness of the tubular support structure.

In another embodiment, the plurality of mechanical property modulating elements comprises one or more sets of mechanical property modulating elements. Each set of mechanical property modulating elements is axially spaced apart along the frame structure, and the set(s) of mechanical property modulating elements are configured for modulating the radially isotropic bending stiffness of the tubular support structure in response to laterally deflecting the tubular support structure respectively in the one or more bending directions.

In still another embodiment, the set(s) of mechanical property modulating elements comprise a plurality of sets of mechanical property modulating elements circumferentially offset from each other around the patterned frame structure, and the bending direction(s) comprise a plurality of different bending directions. In this embodiment, two of the sets of mechanical property modulating elements may be circumferentially offset from each other by one hundred eighty degrees. In this case, each of the radially anisotropic secondary bending stiffnesses may have at least one relatively low magnitude circumferential region and at least one relatively high magnitude circumferential region. The magnitude of each of the relatively low magnitude circumferential region(s) may be equal to the magnitude of the primary bending stiffness or higher than the magnitude of the primary bending stiffness. Each of the relatively low magnitude circumferential region(s) may be at a circumferential location of the patterned frame structure where a set of the plurality of mechanical property modulating elements is absent, and each of the relatively high magnitude circumferential region(s) may be centered at a circumferential location of the patterned frame structure where a set of the plurality of mechanical property modulating elements is present. Each of the relatively low magnitude circumferential region(s) may be centered at a circumferential location of the patterned frame structure where a first set of the plurality of mechanical property modulating elements is present, and each of the relatively high magnitude circumferential region(s) may be centered at a circumferential location of the patterned frame structure where a second set of the plurality of mechanical property modulating elements is present, in which case, the first and second sets of mechanical property elements may modulate the patterned frame structure in different manners.

The present inventions also comprise a method of distally advancing an elongate intravascular medical device (e.g., a guidewire, a catheter, a guide sheath, or an intravascular implant delivery wire) within a vasculature of a patient. The method comprises introducing the elongate intravascular medical device within the vasculature of the patient, distally advancing a lengthwise portion of the elongate intravascular medical device within a first bend in the vasculature of the patient. The lengthwise portion may be, e.g., a distal end of the elongate intravascular medical device or may be proximal to the distal end of the elongate intravascular medical device. The method further comprises distally advancing the lengthwise portion of the elongate intravascular medical device within a second bend in the vasculature of the patient. The second bend has a curvature higher than a curvature of the first bend. The method further comprises distally advancing the elongate intravascular medical device within the vasculature of the patient until a distal end of the elongate intravascular medical device is located at a target site within the vasculature of the patient. One method may further comprise performing an additional medical procedure at the target site using the elongate intravascular medical device.

In accordance with a fifth aspect of the present inventions, the lengthwise portion of the elongate intravascular medical device has a primary bending stiffness while be distally advanced within the first bend in the vasculature of the patient. The method further comprises transitioning the primary bending stiffness of the lengthwise portion of the elongate intravascular medical device to a secondary bending stiffness greater than the primary bending stiffness in response to the distal advancement of the lengthwise portion of the elongate intravascular medical device within the second bend. For example, a highest magnitude of the secondary bending stiffness may be less than 500% of the primary bending stiffness, and preferably, less than 200% of the primary bending stiffness.

In one method, the lengthwise portion of the elongate intravascular medical device has a patterned frame structure that provides the primary bending stiffness and secondary bending stiffness to the elongate intravascular medical device, and the elongate intravascular medical device has mechanical property modulating elements axially spaced apart along the patterned frame structure. In this case, the mechanical property modulating elements are configured for transitioning the primary bending stiffness to the secondary bending stiffness in response to the distal advancement of the lengthwise portion of the elongate intravascular medical device within the second bend.

Another method further comprises distally advancing the lengthwise portion of the elongate intravascular medical device within a third bend in the vasculature of the patient. The third bend has a curvature lower than the curvature of the second bend, in which case, the method further comprises transitioning secondary bending stiffness of the lengthwise portion of the elongate intravascular medical device to the primary bending stiffness in response to the distal advancement of the lengthwise portion of the elongate intravascular medical device within the third bend. This method may further comprise distally advancing the lengthwise portion of the elongate intravascular medical device within a fourth bend in the vasculature of the patient. The fourth bend has a curvature higher than the curvature of the first bend, and different from the curvature of the second bend, in which case, the method further comprises transitioning the primary bending stiffness of the lengthwise portion of the elongate intravascular medical device to another bending stiffness different from the secondary bending stiffness in response to the distal advancement of the lengthwise portion of the elongate intravascular medical device through the fourth bend.

Still another method further comprises pulling the elongate intravascular medical device, and transitioning a primary tensile stiffness of the lengthwise portion of the elongate intravascular medical device to a secondary tensile stiffness greater than the primary tensile stiffness in response to the pulling of the elongate intravascular medical device.

In yet another method, the secondary bending stiffness of the lengthwise portion of the elongate intravascular medical device is radially anisotropic, such that the secondary bending stiffness has a relatively low magnitude circumferential region and a relatively high magnitude circumferential region. This method further comprises rotating the elongate intravascular medical device about its longitudinal axis, such that the primary bending stiffness of the lengthwise portion of the elongate intravascular medical device transitions to the relatively high magnitude circumferential region of the secondary bending stiffness when the lengthwise portion of the rotated intravascular medical device is distally advanced within the second bend. In this method, the magnitude of the relatively low magnitude circumferential region of the secondary bending stiffness is equal to the magnitude of the primary bending stiffness or higher than the magnitude of the primary bending stiffness. This method may further comprise, prior to rotating the elongate intravascular medical device about its longitudinal axis, distally advancing the lengthwise portion of the elongate intravascular medical device into the second bend, while having the relatively low magnitude circumferential region of the secondary bending stiffness, such that the lengthwise portion of the elongate intravascular medical device cannot be successfully distally advanced through the second bend, and proximally retracting the lengthwise portion of the elongate intravascular medical device.

In accordance with a sixth aspect of the present invention, the lengthwise portion of the elongate intravascular medical device has a radially isotropic bending stiffness while be distally advanced within the first bend in the vasculature of the patient. The method further comprises transitioning the radially isotropic bending stiffness of the lengthwise portion of the elongate intravascular medical device to a radially anisotropic bending stiffness in response to the distal advancement of the lengthwise portion of the elongate intravascular medical device within the second bend.

In one method, the lengthwise portion of the elongate intravascular medical device has a patterned frame structure that provides the radially isotropic bending stiffness to the elongate intravascular medical device, and the elongate intravascular medical device has mechanical property modulating elements axially spaced apart along the patterned frame structure. The mechanical property modulating elements are configured for transitioning the radially isotropic bending stiffness to the radially anisotropic bending stiffness in response to the distal advancement of the lengthwise portion of the elongate intravascular medical device within the second bend.

Another method further comprises distally advancing the lengthwise portion of the elongate intravascular medical device within a third bend in the vasculature of the patient, the third bend having a curvature lower than the curvature of the second bend, and transitioning the radially anisotropic bending stiffness of the lengthwise portion of the elongate intravascular medical device to the radially isotropic bending stiffness in response to the distal advancement of the lengthwise portion of the elongate intravascular medical device within the third bend. In this method, the radially anisotropic bending stiffness may have a relatively low magnitude circumferential region and a relatively high magnitude circumferential region, in which case, the method may further comprise rotating the elongate intravascular medical device about its longitudinal axis, such that the radially isotropic bending stiffness of the lengthwise portion of the elongate intravascular medical device transitions to the relatively high magnitude circumferential region of the radially anisotropic bending stiffness when the lengthwise portion of the rotated intravascular medical device is distally advanced within the second bend. In this method, the magnitude of the relatively low magnitude circumferential region of the secondary bending stiffness is equal to the magnitude of the primary bending stiffness or higher than the magnitude of the primary bending stiffness. This method may further comprise, prior to rotating the elongate intravascular medical device about its longitudinal axis, distally advancing the lengthwise portion of the elongate intravascular medical device into the second bend, while having the relatively low magnitude circumferential region of the secondary bending stiffness, such that the lengthwise portion of the elongate intravascular medical device cannot be successfully distally advanced through the second bend, and proximally retracting the lengthwise portion of the elongate intravascular medical device.

Other and further aspects and features of embodiments will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of one embodiment of a guidewire constructed in accordance the present inventions, particularly showing a distal section of the guidewire in a straight configuration;

FIG. 2 is a plan view of the guidewire of FIG. 1, particularly showing a distal section of the guidewire in a curved configuration;

FIG. 3 is a longitudinal-sectional view of the distal section of the guidewire of FIG. 1;

FIG. 4 is a plan view of one embodiment of a catheter constructed in accordance the present inventions, particularly showing a distal section of the catheter in a straight configuration;

FIG. 5 is a plan view of the catheter of FIG. 4, particularly showing a distal section of the catheter in a curved configuration;

FIG. 6 is a longitudinal-sectional view of the distal section of the catheter of FIG. 4;

FIG. 7 is a profile view of one embodiment of a tubular support structure used in the guidewire of FIGS. 1-3 or the catheter of FIG. 4-6, particularly having four circumferentially aligned sets (columns) of mechanical property modulating elements;

FIG. 8 is a profile view of the tubular support structure of FIG. 7, particularly showing lateral deflection of the tubular support structure;

FIG. 9 is a profile view of the tubular support structure of FIG. 7, particularly showing the tubular support structure having an axially stretched configuration;

FIG. 10 is an axial view of the tubular support structure of FIG. 7;

FIG. 11 is a plan view of a radially isotropic primary bending stiffness and a radially isotropic secondary bending stiffness of the tubular support structure of FIG. 7;

FIG. 12 is a profile view of another embodiment of a tubular support structure used in the guidewire of FIGS. 1-3 or the catheter of FIG. 4-6, particularly having four circumferentially misaligned sets of mechanical property modulating elements;

FIG. 13 is a profile view of still another embodiment of a tubular support structure used in the guidewire of FIGS. 1-3 or the catheter of FIG. 4-6, particularly having two circumferentially aligned sets (columns) of mechanical property modulating elements;

FIG. 14 is an axial view of the tubular support structure of FIG. 13;

FIG. 15 is a plan view of a radially isotropic primary bending stiffness and a radially anisotropic secondary bending stiffness of the tubular support structure of FIG. 13;

FIG. 16 is a profile view of yet another embodiment of a tubular support structure used in the guidewire of FIGS. 1-3 or the catheter of FIG. 4-6, particularly having a single circumferentially aligned set (column) of mechanical property modulating elements;

FIG. 17 is an axial view of the tubular support structure of FIG. 16;

FIG. 18 is a plan view of a radially isotropic primary bending stiffness and a radially anisotropic secondary bending stiffness of the tubular support structure of FIG. 16;

FIG. 19 is a plan view of a radially isotropic primary bending stiffness and a radially anisotropic secondary bending stiffness of the tubular support structure of FIG. 7;

FIG. 20 is a diagram illustrating uniform (primary) bending stiffness and progressive (secondary) bending stiffnesses plotted against lateral deflection of the tubular support structure of FIG. 7;

FIG. 21 is a diagram illustrating uniform (primary) tensile stiffness and progressive (secondary) tensile stiffnesses plotted against axial stretch of the tubular support structure of FIG. 7;

FIG. 22A is a close-up view of a mechanical property modulating element and a patterned frame structure of the tubular support structure of FIG. 7, particularly showing the positional relationship between the mechanical property modulating element and the patterned frame structure when the tubular support structure is relaxed;

FIG. 22B is a close-up view of the mechanical property modulating element and the patterned frame structure of FIG. 22A, particularly showing the positional relationship between the mechanical property modulating element and the patterned frame structure when the tubular support structure is laterally deflected or axially stretched;

FIG. 23 is a perspective view of one specific embodiment of the tubular support structure of FIG. 7;

FIG. 24 is another perspective view of the tubular support structure of FIG. 23;

FIG. 25 is a profile view of the tubular support structure of FIG. 23;

FIG. 26 is a profile view of the tubular support structure of FIG. 25, particularly showing lateral deflection of the tubular support structure;

FIG. 27 is a partially cutaway perspective view of the tubular support structure of FIG. 23;

FIG. 28A is a close-up view of one embodiment of a mechanical property modulating element of the tubular support structure of FIG. 23, particularly showing the positional relationship between the mechanical property modulating element and the patterned frame structure when the tubular support structure is relaxed;

FIG. 28B is a close-up view of the mechanical property modulating element of FIG. 28A, particularly showing the positional relationship between the mechanical property modulating element and the patterned frame structure when the tubular support structure is laterally deflected or axially stretched;

FIG. 29 is a profile view of another specific embodiment of the tubular support structure of FIG. 7;

FIG. 30 is a perspective view of still another specific embodiment of the tubular support structure of FIG. 7;

FIG. 31 is a partially cutaway perspective view of the tubular support structure of FIG. 30;

FIG. 32 is a perspective view of yet another specific embodiment of the tubular support structure of FIG. 7;

FIG. 33 is a perspective view of yet another specific embodiment of the tubular support structure of FIG. 7;

FIG. 34 is a profile view of the tubular support structure of FIG. 33;

FIG. 35 is a perspective view of yet another specific embodiment of the tubular support structure of FIG. 7;

FIG. 36 is a profile view of the tubular support structure of FIG. 35;

FIG. 37A is a close-up view of another embodiment of a mechanical property modulating element of the tubular support structure of FIG. 23, particularly showing the positional relationship between the mechanical property modulating element and the patterned frame structure when the tubular support structure is relaxed;

FIG. 37B is a close-up view of the mechanical property modulating element of FIG. 37A, particularly showing the positional relationship between the mechanical property modulating element and the patterned frame structure when the tubular support structure is laterally deflected or axially stretched in one direction;

FIG. 37C is a close-up view of the mechanical property modulating element of FIG. 37A, particularly showing the positional relationship between the mechanical property modulating element and the patterned frame structure when the tubular support structure is laterally deflected or axially stretched in another direction;

FIG. 38 is a flow diagram illustrating one method of using an elongate intravascular medical device that incorporates the tubular support structure of FIG. 7 within a vasculature of a patient;

FIGS. 39A-39J are plan views illustrating the use of the elongate intravascular medical device within the vasculature of the patient in accordance with the method of FIG. 38;

FIG. 40 is a flow diagram illustrating another method of using an elongate intravascular medical device that incorporates the tubular support structure of FIG. 7 within a vasculature of a patient; and

FIGS. 41A-41H are plan views illustrating the use of the elongate intravascular medical device within the vasculature of the patient in accordance with the method of FIG. 40.

### DETAILED DESCRIPTION OF THE ILLUSTRATED EMBODIMENTS

This disclosure describes a tubular support structure in the form of a slotted hypotube that can be incorporated into an elongated intravascular medical device (e.g., the distal end, the proximal end, and/or any region therebetween) which can be navigated through a tortuous and intricate vasculature of a patient. The tubular support structure may be scaled to be incorporated into any size of an intravascular medical device from a guidewire, to a working or diagnostic catheter, all the way up to a guide sheath. The tubular support structure may also be incorporated into any movable component in an intravascular medical device, e.g., an intravascular implant delivery wire. As will be described in further detail below, the mechanical properties of the tubular support structure are dynamically modulated in response to laterally deflecting and/or axially stretching the tubular support structure. In this manner, an intravascular medical device in which the tubular support structure is incorporated will assume the dynamically modulated mechanical properties of the tubular support structure.

For example, the bending stiffness of the tubular support structure increases in response to laterally deflecting the tubular support structure, while tensile stiffness of the tubular support structure increases in response to axially stretching the tubular support structure, via unique features of the tubular support structure. Thus, an intravascular medical device that incorporates the tubular support structure may have a relatively low primary bending stiffness, such that it can be more easily advanced through low to moderate bends in the vasculature of the patient with low tracking force, while also having a relatively high secondary bending stiffness to prevent prolapse when advanced through high bends in the vasculature of the patient.

Furthermore, the tensile stiffness, and thus the tensile strength, of such tubular support structure will increase as the elongate intravascular medical device is axially stretched, thereby resisting deformation of the elongate intravascular medical device in response to the application of significant tensile forces on the elongate intravascular medical device. The tubular support structure can be self-limiting in terms of how much bending moment can be applied to lateral ribs of struts, thereby increasing the tensile strength for a given initial bending stiffness. Thus, the tubular support structure can be composed of a more flexible material without sacrificing the tensile strength of the tubular support structure, such that an intravascular medical device that incorporates the tubular support structure can have both a relatively high bending flexibility and a relatively high tensile strength contrary to the belief that these two countervailing mechanical properties must be balanced, and thus, compromised.

The bending stiffness of the tubular support structure can also be modulated, such that it has a radially isotropic primary bending stiffness, but a radially anisotropic secondary bending stiffness. In this manner, the bending stiffness of the elongate intravascular medical device that incorporates the tubular support structure may be selected when traversing bends in the vasculature of the patient by rotating the elongate intravascular medical device about its axis. For example, the lower bending stiffness for the elongate intravascular medical device may be selected in an attempt to traverse a high bend in the vasculature of the patient, and if such attempt fails (e.g., a prolapse event occurs), the elongate intravascular medical device may be rotated to increase its bending stiffness, and an attempt to traverse the high bend in the vasculature of the patient can be repeated.

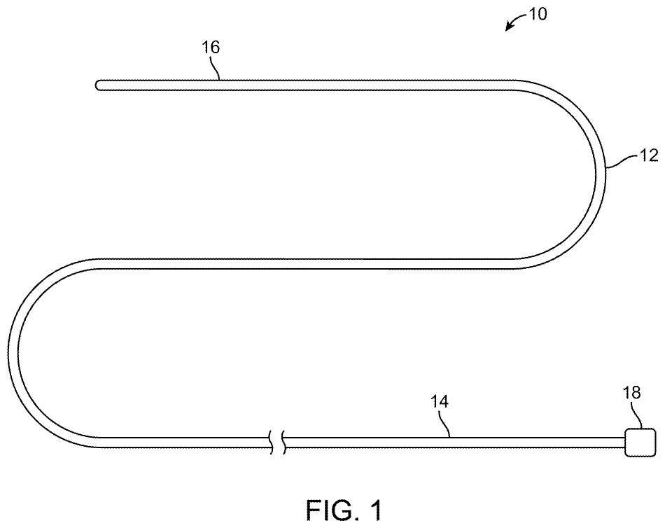

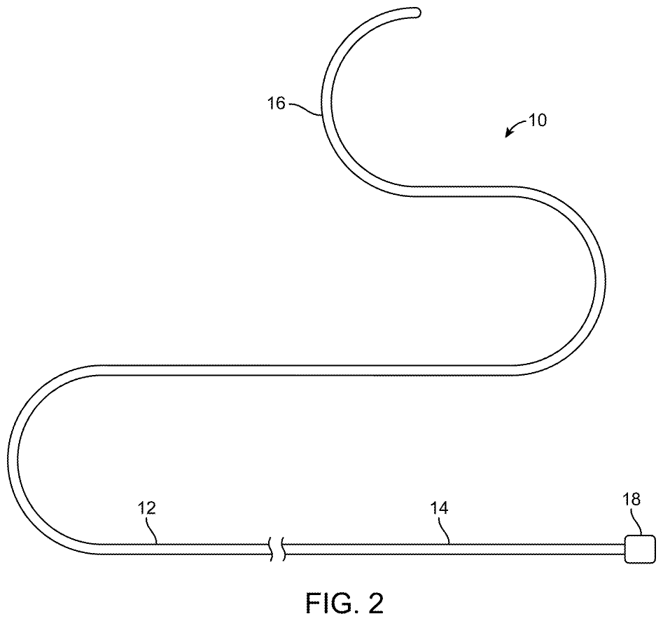

Referring to FIGS. 1-2, one embodiment of an intravascular guidewire 10 that incorporates the aforementioned tubular support structure will now be described. They may be used for intravascular procedures, e.g., in conjunction with another medical device, which may take the form of a catheter, to treat and/or diagnostic a medical condition within a patient. Of course, as one alternative example, the guidewire 10 may be used in any one of a variety of manners within the vasculature of a patient. For example, the guidewire 10 may be configured for delivering an implant (not shown), in which case, the guidewire 10 may function as a delivery wire or a pushwire slidably disposed within the lumen of a delivery catheter. As another alternative example, the guidewire 10 may be used to cross an occlusion or stenosis in the vasculature of the patient. The guidewire 10 may be suitable for use in neurological interventions, coronary interventions, peripheral interventions, etc.

The guidewire 10 generally comprises an elongated guidewire body 12 having a proximal section 14 and a distal section 16. The free end of the proximal section 14 of the guidewire body 12 remains outside of the patient and accessible to an operator (e.g., clinician or physician), while the remainder of the guidewire body 12, including the distal section 16, is sized and dimensioned to reach remote locations of the vasculature of the patient. A torquer 18 can be affixed to the free end of the proximal section 14 of the guidewire body 12 to torque the guidewire 10 during a medical procedure. The torquer 18 is shaped to be ergonomically grasped by the thumb and forefinger of the operator and manipulated to push, pull, or rotate the guidewire body 12. The torquer 18 may be repositioned as necessary as the guidewire 10 is advanced through the vasculature of the patient.

The guidewire body 12 has a suitable length for accessing a target tissue site within the patient from a vascular access point. The target tissue site depends on the medical procedure for which the guidewire 10 is used. As such, the size of the guidewire 10 may be appropriately sized for any given intervention. For example, the guidewire 10 may have a suitable length (e.g., 100-450 cm) and a suitable diameter (e.g., 1F-3F). In one embodiment, the outer diameter of the guidewire body 12 may be uniform along the length of the guidewire body 12. In another embodiment, the outer diameter of the guidewire body 12 may taper in either a gradual fashion or a step-wise fashion from a first outer diameter of the proximal section 14 to a second outer diameter at the distal section 16 to facilitate navigation in tortuous vasculature. Although depicted as having a generally round cross-sectional shape, it can be appreciated that the guidewire body 12 can include other cross-sectional shapes or combinations of shapes, e.g., oval, rectangular, triangular, polygonal, and the like.

The guidewire body 12 has a linear configuration that is relatively straight at room and/or body temperature, yet flexible to bend when subject to external forces, such that the guidewire body 12 may be advanced through the vasculature of the patient. The guidewire body 12 has varying stiffness sections from higher stiffness at the proximal section 14, so that it has sufficient pushability to advance through the patient's vascular system and torqueability to transmit rotational force to the distal section 16, while gradually reducing stiffness to a lower stiffness along the distal section 16, so that it can easily transition between a straight configuration (FIG. 1) and a curved configuration (FIG. 2).

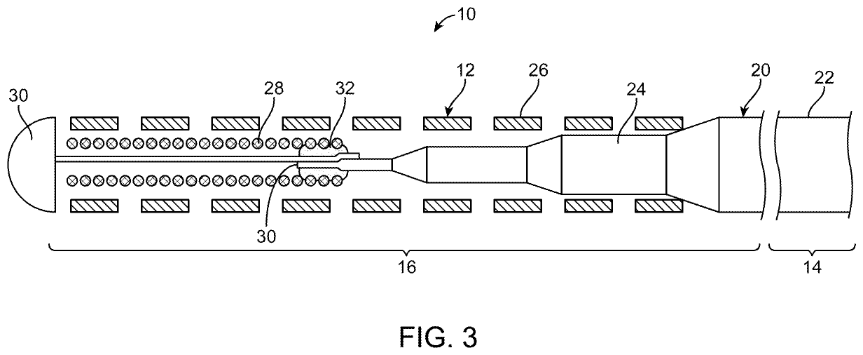

In particular, referring specifically to FIG. 3, the guidewire body 12 comprises a core wire 20 having a proximal section 22 and the distal section 24, a tubular support structure 26 affixed over the distal section 24 of the core wire 20, a radiopaque coil 28 affixed within the tubular support structure 26 to the distal section 24 of the core wire 20, and an atraumatic distal tip member 30 affixed to the distal tip 30 of the core wire 20 and/or tubular support structure 26 via a soldering bond 32. The radiopaque coil 28 may be composed of a suitable radiopaque material, e.g., gold, platinum, palladium, tantalum, tungsten alloy, polymer material loaded with a radiopaque filler, and the like. The distal tip member 30 may be, e.g., a solder ball or may take the form of a polymeric tip disposed at the end of the core wire 20.

The tubular support structure 26 has a slot pattern designed to enhance the flexibility of the guidewire body 12, while still allowing for suitable torque transmission characteristics. In the illustrated embodiment, the slot pattern is provided along substantially the entire length, and around the entire circumference, of the tubular support structure 26, although alternatively, the slot pattern may be provided along any lengthwise portion or circumferential portion of the tubular support structure 26. While the tubular support structure 26 is illustrated as being located in the distal section 16 of the guidewire body 12, it should be appreciated that the tubular support structure 26 may be located anywhere in the guidewire body 12, including the proximal section 14 of the guidewire body 12, where it is desired to dynamically modulate the mechanical properties (and in particular, the bending stiffness and pullability) of the guidewire body 12. Further details of the structure and function of tubular support structures that can be used as the tubular support structure 26 of the guidewire body 12 will be described in further detail below.

In the illustrated embodiment, the core wire 20 is a unitary member. The distal section 24 of the core wire 20 includes gradually reducing tapered sections, such that the flexibility of the distal section 16 of the guidewire body 12 gradually increases. The tapered sections of the distal section 24 of the core wire 20 may be formed by a number of different techniques, e.g., by centerless grinding methods, stamping methods, and the like. The core wire 20 may be composed of a metal, metal alloy, polymer, metal-polymer composite, etc. In alternative embodiments, the proximal section 22 and the distal section 24 of the core wire 20 may be composed of different materials (e.g., materials having different moduli of elasticity), resulting in a different in flexibility), in which case, a connector (not shown) may couple the proximal section 22 and distal section 24 of the core wire 20 together via welding, brazing, adhesive, or the like. The proximal section 22 of the core wire 20 may be composed of a material (e.g., straightened 304v stainless steel), such that it is relatively stiff for pushability and torqueability, while the distal section 24 of the core wire 20 may be composed of a material (e.g., straightened super elastic or linear elastic alloy, e.g., nickel-titanium alloy), such that it is relatively flexible by comparison for better lateral trackability and steerability.

In the illustrated embodiment, the guidewire body 12 has an isotropic primary bending stiffness (i.e., it has a flexibility in bending that is substantially equal in all radial directions). For example, the distal section 24 of the core wire 20 may have a circular-section and the tubular support structure 26 may have a circumferentially uniform slotted pattern. In alternative embodiments, the guidewire body 12 includes one or more structural features that allows it to have an anisotropic primary bending stiffness. For example, at least a portion of the distal section 24 of the core wire 20 may be flattened and/or the tubular support structure 26 may have a circumferentially non-uniform slotted pattern. As such, the guidewire body 12 may have one or more preferred bending directions or otherwise may be more easily bend in on direction than in another. In some embodiments, the preferred bending direction is oriented in only a single radial direction along one side of the guidewire body 12. For example, if the preferred bending direction points only to the left of the guidewire body 12 (as illustrated), the guidewire body 12 may be more flexible in bending when bending to the left than in any other direction (including, e.g., directions perpendicular or orthogonal to the preferred bending-direction). In other embodiments, the preferred bending direction may be oriented in opposite radial directions along opposite sides of the guidewire body 12. For example, if the preferred bending direction points to both the left and the right of the guidewire body 12 (as illustrated), the guidewire body 12 may be more flexible in bending to the left or right than in any other direction (including, e.g., directions perpendicular or orthogonal to the preferred bending direction).

In one embodiment, the guidewire body 12 further comprises an outer polymer jacket (not shown) disposed over portions of the core wire 20 and/or tubular support structure 26, thereby defining a generally smooth outer surface for the guidewire 10 and/or tubular support structure 26. In other embodiments, however, such an outer polymer jacket may be absent from a portion of all of the guidewire body 12, such that the core wire 20 and/or tubular support structure 26 may form the outer surface of the guidewire body 12. In some embodiments, the exterior surface of the core wire 20 and/or tubular support structure 26 may be sandblasted, beadblasted, sodium bicarbonate-blasted, electropolished, etc. In some embodiments, at least a portion of the outer surface of the guidewire body 12 (e.g., the outer surface of the outer polymer jacket (if provided) or the other surfaces of the core wire 20 and/or tubular support structure 26 (if an outer polymer jacket is not provided)) includes one or more coatings, such as, e.g., an anti-thrombogenic coating, which may help reduce the formation of thrombi in vitro, an anti-microbial coating, or a lubricating coating (e.g., a hydrophilic coating), which may reduce static friction or kinetic friction between the guidewire body 12 and tissue of the patient as the guidewire body 12 is advanced through the vasculature or through another catheter.

Although the tubular support structure 26 has been described as being incorporated into an elongated intravascular medical device in the form of a guidewire 10 for the purpose of dynamically modulating the mechanical properties of the guidewire body 12, it should be appreciated that the tubular support structure 26 may be incorporated into any suitable elongated intravascular device for the purpose of dynamically modulating the mechanical properties of that device.

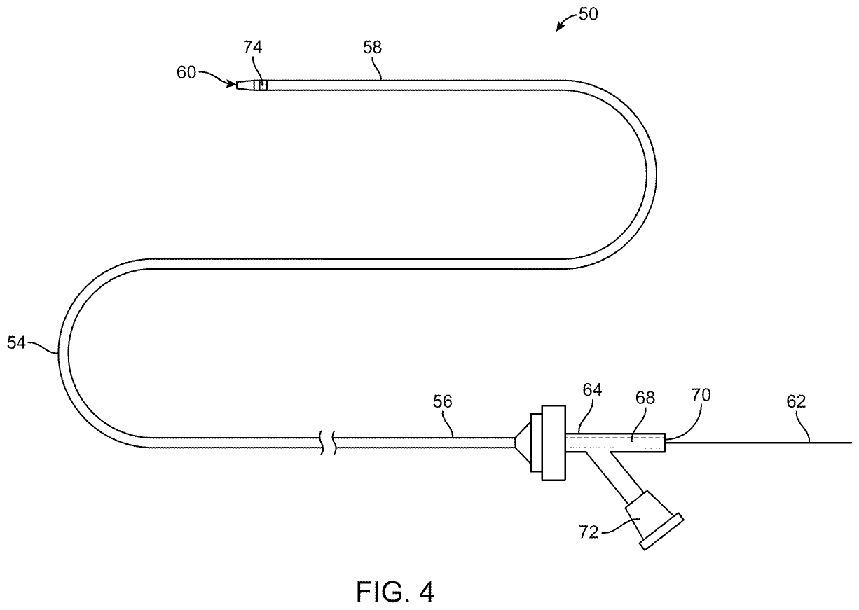

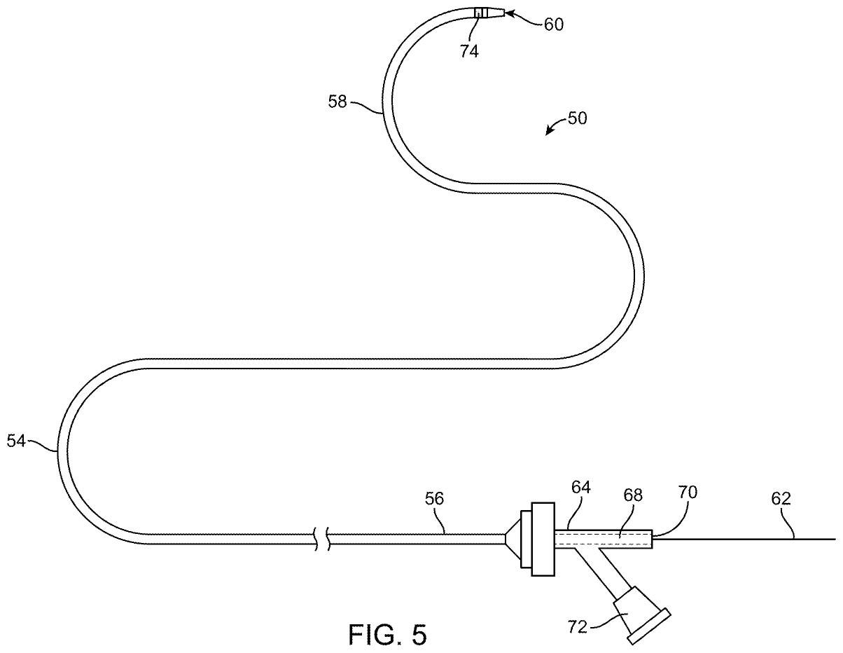

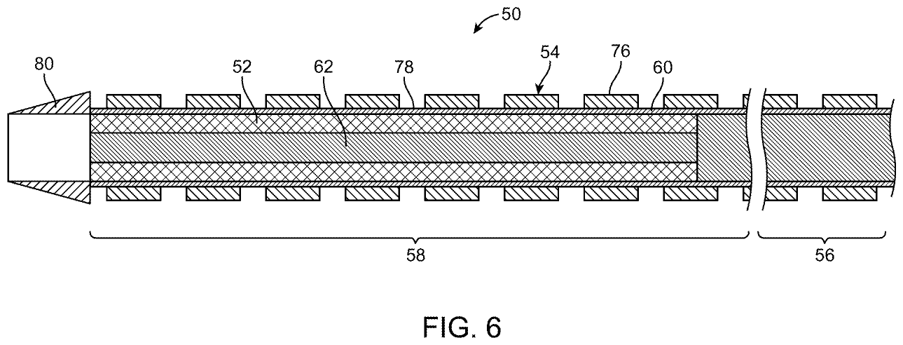

For example, with reference now to FIGS. 4-5, one embodiment of an intravascular catheter 50 will now be described. In the illustrated embodiment, the intravascular catheter 50 serves as a delivery catheter for delivering an intravascular implant 52 (e.g., a stent, stent graft, flow-diverter, vaso-occlusive device, vena cava filter, etc.) (shown in FIG. 6) at a target site within the vasculature of a patient, although alternative embodiments of the intravascular catheter 50 may deliver other medical devices, e.g., another catheter, a guide member, a thrombectomy device, etc. Furthermore, other alternative embodiments of the intravascular catheter 50 may serve as a diagnostic catheter or another type of a treatment catheter (e.g., an access catheter, a balloon catheter, atherectomy catheter, drug delivery catheter, etc.).

The intravascular catheter 50 generally comprises an elongated sheath body 54 topologically divided between a proximal section 56 and a distal section 58, an inner sheath lumen 60 extending within the sheath body 54, a pusher member 62 slidably disposed within the sheath lumen 60, and a proximal adapter 64 affixed to the free end of the proximal section 56 of the sheath body 54.

The diameter of the inner sheath lumen 60 may vary based on the medical procedure for which the intravascular catheter 50 is used, and in the illustrated embodiment, is sized to accommodate the intravascular implant 52. The diameter of the inner sheath lumen 60 may be substantially constant from the proximal section 56 of the sheath body 54 to the distal section 58 of the sheath body 54 or may taper from a first diameter at the proximal section 56 of the sheath body 54 to a second different diameter at the distal section 58 of the sheath body 54. The inner sheath lumen 60 terminates in a distal port 66 at the end of the distal section 58 of the sheath body 54.

The pusher member 62 carries the intravascular implant 52 and can be distally advanced within the inner sheath lumen 60 to deploy the intravascular implant 52 from the intravascular catheter 50 at the target site of the vasculature of the patient. The proximal adapter 64 is affixed to the proximal section 56 of the intravascular catheter 50 using suitable means, e.g., adhesive, welding, etc. The proximal adapter 64 comprises a central bore 68 (shown in phantom) in communication with the inner sheath lumen 60. The central bore 68 terminates in a proximal port 70 for allowing loading of the pusher member 62 and intravascular implant 12 into the intravascular catheter 50. The proximal adapter 64 further comprises a side port 72 in fluid communication with the central bore 68 for introducing fluids into the inner sheath lumen 60 in order to hydrate the pusher member 62 and intravascular implant 12. In some embodiments, another structure (not shown) in addition to, or instead of, the proximal catheter hub 64 may be affixed to the proximal section 56 of the intravascular catheter 50. The intravascular catheter 50 further comprises one or more radiopaque marker bands 74 (only one shown) disposed on the distal section 58 of the sheath body 54 proximate the distal port 66, such that the location of the distal tip of the intravascular catheter 50 within the patient's vasculature system, or relative to the partially or fully deployed intravascular implant 12, can be identified using medical imaging technology (e.g., fluoroscopy). The radiopaque band(s) 74 may be composed of a suitable radiopaque material, e.g., gold, platinum, palladium, tantalum, tungsten alloy, polymer material loaded with a radiopaque filler, and the like.

The free end of the proximal section 56 of the sheath body 54 remains outside of the patient and accessible to an operator (e.g., clinician or physician), while the remainder of the sheath body 54, including the distal section 58, is sized and dimensioned to reach remote locations of the vasculature of the patient. The sheath body 54 has a suitable length for accessing a target tissue site within the patient from a vascular access point. The target tissue site depends on the medical procedure for which the intravascular catheter 50 is used. For example, if the intravascular catheter 50 is used to access vasculature in a brain of a patient from a femoral artery access point at the groin of the patient, the overall length of the sheath body 54 may be 125 cm-200 cm. The outer diameter of the sheath body 54 may be in the range of 3F-10F. In one embodiment, the outer diameter of the sheath body 54 may be uniform along the length of the catheter body 18. In another embodiment, the outer diameter of the sheath body 54 may taper in either a gradual fashion or a step-wise fashion from a first outer diameter of the proximal section 56 to a second outer diameter at the distal section 58 to facilitate navigation in tortuous vasculature. Although depicted as having a generally round cross-sectional shape, it can be appreciated that the sheath body 54 can include other cross-sectional shapes or combinations of shapes, e.g., oval, rectangular, triangular, polygonal, and the like.

The sheath body 54 has a linear configuration that is relatively straight at room and/or body temperature, yet flexible to bend when subject to external forces, such that the sheath body 54 may be advanced through the vasculature of the patient. The sheath body 54 has variable stiffness sections from higher stiffness at the proximal section 56, so it has sufficient pushability to advance through the patient's vascular system and torqueability to transmit rotational force to the distal section 58, while gradually reducing stiffness to a lower stiffness along the distal section 58, so that the it can easily transition between a straight configuration (FIG. 4) and a curved configuration (FIG. 5). The sheath body 54 may optionally comprise an intermediate section (not shown) that may gradually transition the relatively high bending stiffness of the proximal section 56 to the relatively low bending stiffness of the distal section 58.

Referring specifically to FIG. 6, the sheath body 54 generally comprises a tubular support structure 76, an inner polymer liner 78 disposed within the tubular support structure 76, and an atraumatic distal tip member 80 affixed to the distal tip of the tubular support structure 76. The sheath body 54 may further comprise a tie layer (not shown) that attaches the inner polymer liner 78 to the tubular support structure 76.

The tubular support structure 76 has a slot pattern designed to enhance the flexibility of the sheath body 54, while still allowing for suitable torque transmission characteristics. In the illustrated embodiment, the slot pattern is provided along substantially the entire length, and around the entire circumference, of the tubular support structure 76, although alternatively, the slot pattern may be provided along any lengthwise portion or circumferential portion of the tubular support structure 76. While the tubular support structure 76 is illustrated as being located along both the proximal section 56 and the distal section 58 of the sheath body 54, it should be appreciated that the tubular support structure 76 may be located anywhere in the sheath body 54, e.g., only the distal section 58 of the sheath body 54, where it is desired to dynamically modulate the mechanical properties (and in particular, the bending stiffness and pullability) of the sheath body 54. Further details of the structure and function of tubular support structures that can be used as the tubular support structure 76 of the sheath body 54 will be described in further detail below.

The inner polymer liner 78 is composed of a low friction material (e.g., polytetrafluoroethylene (PTFE), expanded PTFE (ePTFE, e.g., unidirectional ePTFE or bi-directional ePTFE), a fluoropolymer, perfluoroalkoxy alkane (PFA), fluorinated ethylene polyethylene (FEP), polyethylene (PE), or any combination thereof) that surrounds the inner sheath lumen 60. Thus, the inner polymer liner 78 may provide a lubricious inner surface to facilitate passing the intravascular implant 52 through the inner sheath lumen 60.

In the illustrated embodiment, the sheath body 54 has an isotropic primary bending stiffness (i.e., it has a flexibility in bending that is substantially equal in all radial directions). For example, the tubular support structure 76 may have a circumferentially uniform slotted pattern. In alternative embodiments, the sheath body 54 includes one or more structural features that allows it to have an anisotropic primary bending stiffness. As such, the sheath body 54 may have one or more preferred bending directions or otherwise may be more easily bend in one direction than in another. In some embodiments, the preferred bending direction is oriented in only a single radial direction along one side of the sheath body 54. For example, if the preferred bending direction points only to the left of the sheath body 54 (as illustrated), the sheath body 54 may be more flexible in bending when bending to the left than in any other direction (including, e.g., directions perpendicular or orthogonal to the preferred bending-direction). In other embodiments, the preferred bending direction may be oriented in opposite radial directions along opposite sides of the sheath body 54. For example, if the preferred bending direction points to both the left and the right of the sheath body 54 (as illustrated), the sheath body 54 may be more flexible in bending to the left or right than in any other direction (including, e.g., directions perpendicular or orthogonal to the preferred bending direction).

In one embodiment, the sheath body 54 further comprises an outer polymer jacket (not shown) disposed over the entire tubular support structure 76, or portions thereof, thereby defining a generally smooth outer surface for the tubular support structure 76. In other embodiments, however, such an outer polymer jacket may be absent from a portion of or all of the sheath body 54, such that the tubular support structure 76 may form the outer surface of the sheath body 54. If an outer polymer jacket is disposed over the proximal section 56 of the sheath body 54, the sheath body 54 may additionally include a reinforcement layer, such as a braided layer or coiled layer, to enhance the pushability of the sheath body 54. In some embodiments, the exterior surface of the tubular support structure 76 may be sandblasted, beadblasted, sodium bicarbonate-blasted, electropolished, etc. In some embodiments, at least a portion of the outer surface of the sheath body 54 (e.g., the outer surface of the outer polymer jacket (if provided) or the other surfaces of the tubular support structure 76 (if an outer polymer jacket is not provided)) includes one or more coatings, such as, e.g., an anti-thrombogenic coating, which may help reduce the formation of thrombi in vitro, an anti-microbial coating, or a lubricating coating (e.g., a hydrophilic coating), which may reduce static friction or kinetic friction between the sheath body 54 and tissue of the patient as the sheath body 54 is advanced through the vasculature over a guidewire or through a guide sheath. The distal tip member 80 may take the form of a polymeric tip disposed at the end of the core wire 20.

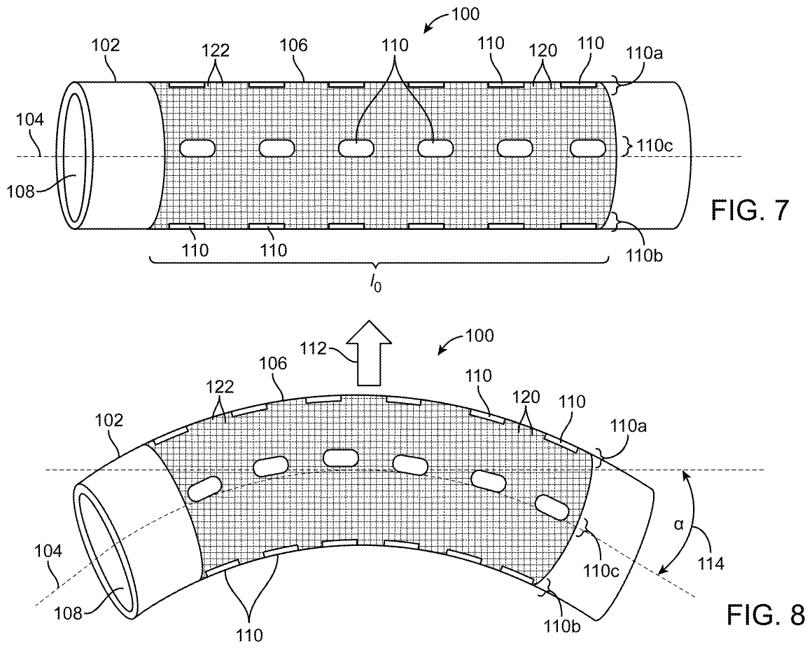

Referring now to FIGS. 7-10, one embodiment of a tubular support structure 100, which can be used for the tubular support structure 26 of the intravascular guidewire 10 illustrated in FIGS. 1-3 or the tubular support structure 76 of the intravascular catheter 50 illustrated in FIGS. 4-6, will be described.

The tubular support structure 100 generally comprises an elongate tubular body 102 having a longitudinal axis 104, a patterned frame structure 106 formed into the tubular body 102, an inner lumen 108 axially disposed along the longitudinal axis 104 of tubular body 102, and a plurality of mechanical property modulating elements 110 axially spaced along and circumferentially spaced around the patterned frame structure 106.

The tubular body 102 may be composed of any variety of suitable materials, e.g., a material that is rigid, but has some flexibility when used to form extremely thin structures, such as the wall of the tubular body 102. In the illustrated embodiment, the tubular body 102 takes the form of a hypotube composed of a metal or a metal alloy (e.g., stainless steel, such as 304 stainless steel, 316 stainless steel, 316L stainless steel, nickel chromium (NiCr) steel, nickel titanium alloy (e.g., nitinol), cobalt/chromium), or the like. Alternatively, the tubular support structure 100 may be composed of a rigid polymer (e.g., polyether-ether ketone (PEEK)). The dimensions of the tubular body 102 may be suitable for one or more desired uses of an elongate medical device, such as the guidewire 10 of FIGS. 1-3 or the intravascular catheter 50 of FIGS. 4-6. As examples, the outer diameter of the tubular body 44 may be in the range of 0.005-0.150 inches. The inner diameter of the tubular body 44 (i.e., the diameter of the inner lumen 108) may, e.g., be in the range of 0.002-0.145 inches.

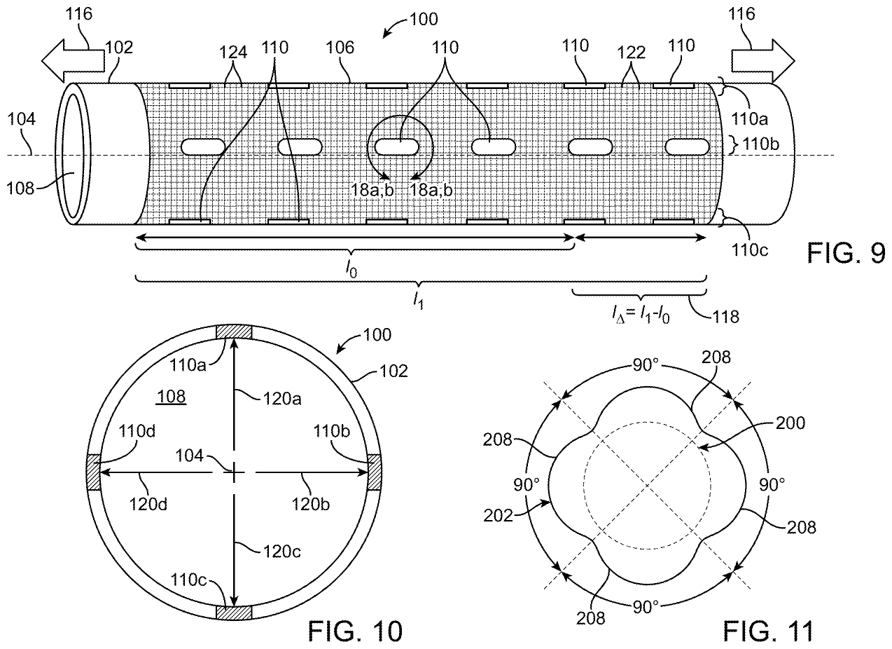

The patterned frame structure 106 enhances the flexibility of the tubular support structure 100, such that, in response to a bending force, it can be laterally deflected in a bending direction 112 from a relaxed configuration (in this case, a straight configuration) (FIG. 7) to a curved configuration (FIG. 8) within a lateral deflection range 114 (a). Although the relaxed configuration of the tubular support structure 100 is illustrated as being a straight configuration, in alternative embodiments, the tubular support structure 100 can be pre-shaped, such that its relaxed configuration is a curve configuration. In this alternative case, the tubular support structure 100 can be laterally deflected, in response to a bending force, in a bending direction within a lateral deflection range from the relaxed curved configuration to an increased curved configuration (i.e., having a radius of curvature less than the radius of curvature of the relaxed curved configuration). In any event, along with the enhanced flexibility of the tubular support structure 100, the patterned frame structure 106, in response to a tensile force 116, will inherently axially stretch in an axial direction 116 from a relaxed length l₀ (FIG. 7) to an increased length l₁ (FIG. 9) within an axial stretch range 118 (l_{Δ}).

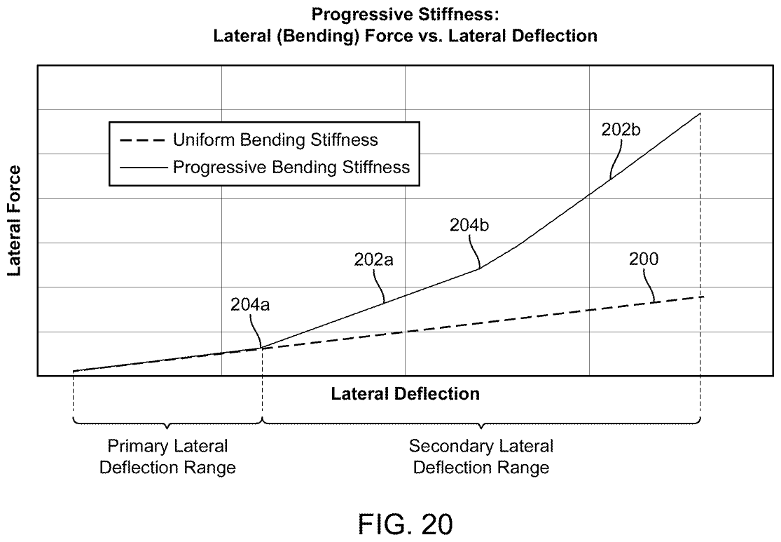

The patterned frame structure 106 may comprise any combination of apertures 122 and members 124 that provide the tubular support structure 100 with a desired primary (or initial) bending stiffness. For example, as illustrated in FIG. 20, the tubular support structure 100 has an exemplary primary bending stiffness 200 (shown by dashed line) that defines a bending force (vertical axis) that linearly varies in direct proportion to the amount of lateral deflection (or in an inverse relationship to the amount of the radius of curvature) (horizontal axis) of the patterned frame structure 106. Thus, as can be seen from FIG. 20, the primary bending stiffness 200 will be, without modulation, generally uniform. The primary bending stiffness 200 is preferably finite (i.e., the primary bending stiffness 200 is preferably substantially greater than zero). For example, the primary bending stiffness 200 may be greater than 0.00001 in²-lb. As will be described in further detail below, the modulating elements 110 modulate the patterned frame structure 106, such that the tubular support structure 100 has one or more secondary bending stiffnesses 202, and thus a progressive bending stiffness.

In the illustrated embodiment, the tubular support structure 100 has a radially isotropic primary bending stiffness 200. For the purposes of this specification, a tubular support structure 100 has a radially isotropic primary bending stiffness 200 if the primary bending stiffness 200 is the same at at least four equally spaced apart radial directions spaced apart. The tubular support structure 100 may achieve an isotropic primary bending stiffness 200 by circumferentially repeating the pattern of apertures 122 and members 124 around the entire patterned frame structure 106. In alternative embodiments, the tubular support structure 100 has an anisotropic primary bending stiffness 200. For example, the primary bending stiffness 200 of the tubular support structure 100 may be lower in first two diametrically opposed radial directions (e.g., radial directions 120a, 120c) than the primary bending stiffness 200 of the tubular support structure 100 in second two diametrically opposed radial directions (e.g., radial directions 120b, 120d) clocked 900 from the first two diametrically opposed radial directions (e.g., radial directions 120a-120b).

Significantly, the modulating elements 110 are configured for modulating, and in particular, incrementally increasing, the bending stiffness of the tubular support structure 100 in response to laterally deflecting the tubular support structure 100 in at least one bending direction, such that the primary bending stiffness 200 of the tubular support structure 100 transitions to one or more higher secondary bending stiffnesses 202, as illustrated in FIG. 20. In particular, the modulation of the bending stiffness of the tubular support structure 100 in response to laterally deflecting the tubular support structure 100 in a particular bending direction will create one or more inflection points 204 that define the transitions between the primary bending stiffness 200 within a primary lateral deflection range and one or more secondary bending stiffnesses 202, and in this case, two secondary bending stiffnesses 202a, 202b, within a secondary lateral deflection range that is greater than the primary lateral deflection range. At each inflection point 204, the bending stiffness of the tubular support structure 100 for the particular bending direction increases. Thus, instead of continuing in an unmodulated manner (as illustrated by the dashed line), the primary bending stiffness 200 of the tubular support structure 100 transitions to the secondary bending stiffnesses 202, and in this case, to the bending stiffness 202a, and then to the bending stiffness 202b. It should be appreciated that the primary bending stiffness 200 of the tubular support structure 100 may transition to any number of secondary bending stiffnesses 202, including only one secondary bending stiffness 202 or more than two secondary bending stiffnesses 202.

The bending stiffness of the tubular support structure 100 may be increased from an initial bending stiffness (in this case, the primary bending stiffness 200) to a maximum bending stiffness (in this case, the highest secondary bending stiffness 202) by less than 500%, and preferably, by less than 200%, such that the trackability of the tubular support structure 100 through the vasculature of the patient is maintained. Additionally, an abrupt change in bending stiffness to a very high value, without modulation (i.e., incrementally increasing), would severely compromise the navigability of the elongate intravascular medical device, and may become too stiff to advance safely within the vasculature of the patient. It should be appreciated that the bending stiffness of the tubular support structure 100 is reversible in that, as the tubular support structure 100 is transitioned back to its straight configuration, the secondary bending stiffness 202b will transition back to the secondary bending stiffness 202a, which will then transition back to the primary bending stiffness 200.

In the illustrated embodiment, the modulating elements 110 are arranged as sets 110a-110d (best shown in FIG. 10), and in this case four circumferentially aligned sets (i.e., in columns), with the modulating elements 110 each of the columns 110a-110d being axially spaced apart from each other along the patterned frame structure 106, and the modulating element columns 110a-110d being circumferentially spaced apart from each other by 90°. In this manner, the bending stiffness of the tubular support structure 100 may be modulated in four different radial directions spaced apart by 90°.

In the illustrated embodiment, only a single modulating element column is responsible (i.e., the single modulating element column will be active while the other three modulating element columns will be inactive) for modulating the bending stiffness of the tubular support structure 100 when laterally deflected in a direction of the respective modulating element column (i.e., the modulating element column that is on the outer edge of the curve (in this case, the modulating element column 110a Illustrated in FIG. 8)).

In an alternative embodiment, the single modulating element column that is responsible for modulating the bending stiffness of the tubular support structure 100 is opposite the direction in which the tubular support structure 100 is laterally deflected (i.e., the modulating element column that is on the inner edge of the curve (in this case, the modulating element column 110c Illustrated in FIG. 8)). In other alternative embodiments, two diametrically opposing modulating element columns are both responsible for modulating the bending stiffness of the tubular support structure 100 when laterally deflected in the direction of one of the two respective modulating element columns (i.e., the modulating element columns that are on the outer edge and inner edge of the curve (in this case, the modulating element columns 110a, 110c Illustrated in FIG. 8)). It should be appreciated that if the tubular support structure 100 is laterally deflected in a direction between a pair of adjacent modulating element columns (e.g., the modulating element column 110a and modulating element column 110b), the respective pair of modulating element columns may contribute to the modulation of the primary bending stiffness of the tubular support structure 100.

It should be appreciated that, because the modulating element columns 110a-110d are circumferentially spaced apart around the patterned frame structure 106 from each other by 90°, the modulating elements 110 may be configured for increasing the bending stiffness of the tubular support structure 100 from an initial radially isotropic primary bending stiffness 200 to one or more radially isotropic secondary bending stiffnesses 202, assuming that the modulating element columns 110a-110d are identical. Thus, the modulating element columns 110a-110d circumferentially modulate the bending stiffness of the tubular support structure 100 in a substantially uniform manner, such that the tubular support structure 100 has radially isotropic secondary bending stiffnesses 202. For the purposes of this specification, a tubular support structure 100 has a radially isotropic secondary bending stiffness 202 if the secondary bending stiffness 202 is the same at at least four equally spaced apart radial directions spaced apart (e.g., four radial directions 120a-120d spaced apart by 90°, as illustrated in FIG. 10). As a result, a secondary bending stiffness 202 may have four 90° circumferential regions 208 respectively centered at the four modulating element columns 110a-110d, as illustrated in FIG. 11.

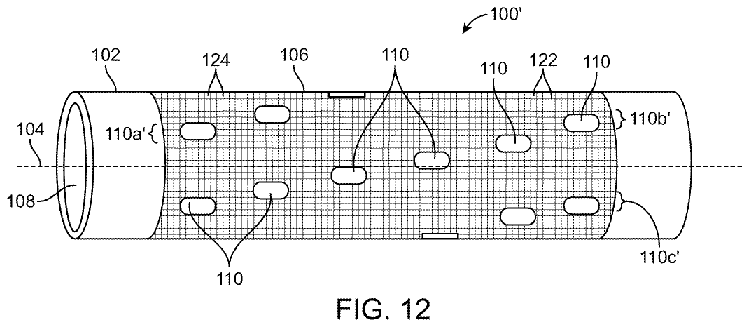

Although the modulating elements 110 are illustrated in FIGS. 7-10 as being arranged in circumferentially aligned sets on the patterned frame structure 106, the modulating elements 110 may be arranged in four circumferentially misaligned sets (only sets 110a′-110c′ shown) on the patterned frame structure 106 in an alternative embodiment of a tubular support structure 100′, as illustrated in FIG. 12. By axially misaligning the modulating elements 110 on the patterned frame structure 106, the secondary bending stiffness 202 of the tubular support structure 100′ may be more radially isotropic. That is, by circumferentially spacing the modulating elements on the patterned frame structure 106 within each modulating element set 110′, any preferential bending direction of the tubular support structure 100′ when in the secondary lateral deflection range that may otherwise result from circumferentially aligned columns of modulating elements or tolerances within the manufacturing process of the tubular support structure 100′ may be smoothed out.

In an alternative embodiment, the modulating elements 110 may be configured for increasing the bending stiffness of the tubular support structure 100 from an initial radially isotropic primary bending stiffness to a radially anisotropic secondary bending stiffness.

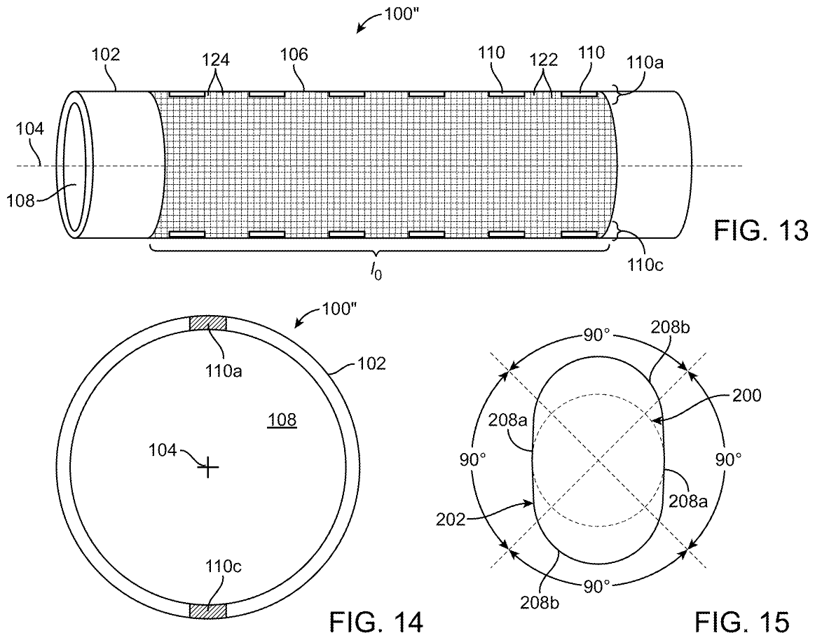

For example, as illustrated in FIGS. 13-14, an alternative embodiment of a tubular support structure 100” may have only two diametrically opposing modulating element columns 110a, 110c disposed on the patterned frame structure 106. In this manner, the tubular support structure 100” has secondary bending stiffnesses 202 when laterally deflected in bending directions towards either of the opposing modulating element columns 110a, 110c and decreased or no secondary bending stiffnesses 202 when laterally deflected at 900 from the opposing modulating element columns 110a, 110c. In this embodiment, the secondary bending stiffness 202 may have two diametrically opposed relatively low magnitude 90° circumferential regions 208a centered at circumferential locations of the patterned frame structure 106 where a modulating element column is absent (i.e., where the modulating element columns 110b, 110d have been omitted), and two diametrically opposed relatively low magnitude 90° circumferential regions 208b centered at circumferential locations of the patterned frame structure 106 where a modulating element column is present (i.e., where the modulating element columns 110a, 110c are present), as illustrated in FIG. 15. The minima of the magnitudes of the relatively low magnitude circumferential regions 208a of the secondary bending stiffness 202 are equal to the magnitude of the primary bending stiffness 200.

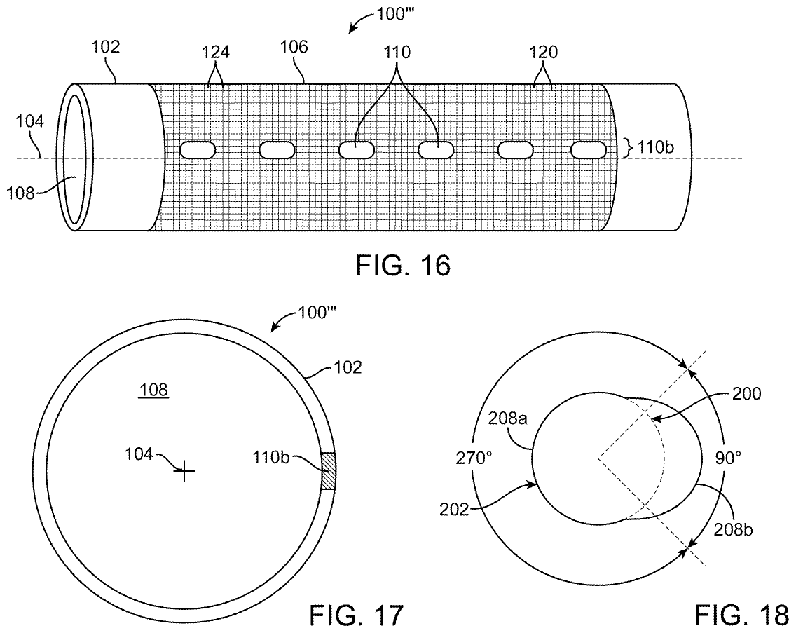

As another example, as illustrated FIGS. 16-17, another alternative embodiment of a tubular support structure 100″′ may have a single modulating element column 110b disposed on the patterned frame structure 106. In this manner, the tubular support structure 100″′ has one or more secondary bending stiffnesses 202 when laterally deflected in a bending direction towards (or alternatively away from) the modulating element column 110b and decreased or no secondary bending stiffnesses 202 when laterally deflected away from (or alternatively toward) the modulating element column 110b. In this embodiment, the secondary bending stiffness 202 may have one relatively low magnitude 270° circumferential region 208a centered at a circumferential location of the patterned frame structure 106 where a modulating element column is absent (i.e., where the modulating element column 110b has been omitted), and one relatively low magnitude 90° circumferential region 208b centered at a circumferential location of the patterned frame structure 106 where a modulating element column is present (i.e., where the modulating element column 110b is present), as illustrated in FIG. 18. The minimum of the magnitude of the relatively low magnitude circumferential region 208a of the secondary bending stiffness 202 is equal to the magnitude of the primary bending stiffness 200.

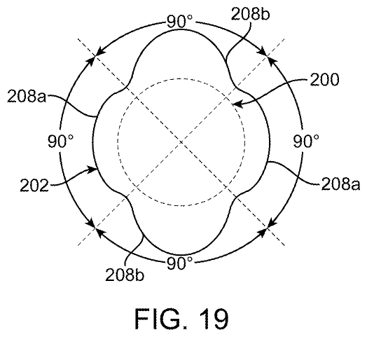

Although the patterned frame structures 106 of the radially isotropic primary bending stiffnesses 200 of the tubular support structures 100″ and 100″′ illustrated in FIGS. 13-14 and 16-17 are configured for being increased to radially anisotropic secondary bending stiffnesses 202 by omitting one or more columns of modulating elements 110, the tubular support structure 100 illustrated in FIGS. 7-10, which has all of the columns of modulating elements 110 circumferentially spaced around the patterned frame structure in a uniform manner, may be designed to increase the radially isotropic primary bending stiffnesses 200 to a radially anisotropic bending stiffness 202. For example, the secondary bending stiffness 202 may have two diametrically opposed relatively low magnitude 90° circumferential regions 208a centered at the modulating element columns 110b, 110d, and two diametrically opposed relatively low magnitude 90° circumferential regions 208b centered at the modulating element columns 110a, 110c, as illustrated in FIG. 19. The minima of the magnitudes of the relatively low magnitude circumferential regions 208a of the secondary bending stiffness 202 are higher than the magnitude of the primary bending stiffness 200.

To provide radial anisotropy to the secondary bending stiffnesses 202 of the tubular support structure 100, at least two of the columns of modulating elements 110 may be designed to modulate the secondary bending stiffnesses 202 in different manners, such that each of the secondary bending stiffnesses 202 is radially anisotropic. For example, all of the modulating elements 110 in one of the columns 110a-110d may modulate the bending stiffness of the tubular support structure 100 for a particular lateral deflection in one radial direction, while none or otherwise less than all of the modulating elements 110 in a different one of the columns 110a-110d may modulate the bending stiffness of the tubular support structure 100 for the same lateral deflection in a different radial direction (e.g., the lengths of the mechanical modulating elements 110 for different columns 110a-110d may differ from each other)). In this case, the secondary bending stiffness 202 illustrated in FIG. 19 may be dynamic in nature in that its contour will change as a function of the magnitude of lateral deflection of the tubular support structure 100. Alternatively, all of the modulating element columns 110a-110d may modulate the bending stiffness of the tubular support structure 100 for the same particular lateral deflection in all radial directions; however, at least two of the modulating element columns 110a-110d may modulate the bending stiffness of the tubular support structure 100 at different magnitudes (e.g., the lengths of the pairs of extensions 172 associated with different ones of the modulating element columns 110a-110d may differ from each other).

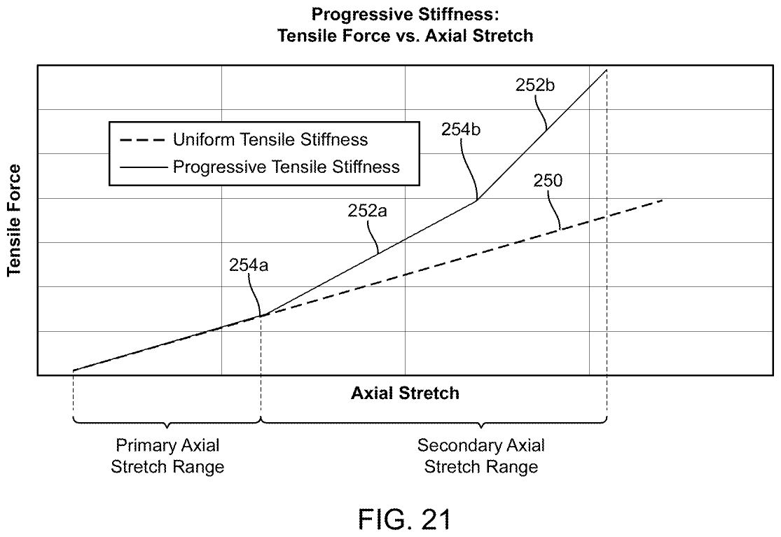

Like the bending stiffness, the combination of apertures 122 and members 124 provides the tubular support structure 100 with a desired primary (or initial) tensile stiffness. For example, as illustrated in FIG. 21, the tubular support structure 100 has an exemplary primary tensile stiffness 250 that defines a tensile stiffness (vertical axis) that linearly varies in direct proportion to the amount of axial stretch (horizontal axis) of the tubular support structure 100. Thus, as can be seen from FIG. 21, the primary tensile stiffness 250 will, without modulation, be generally uniform. The primary tensile stiffness 250 is preferably finite (i.e., the primary tensile stiffness 250 is preferably substantially greater than zero). For example, the primary tensile stiffness 200 may be greater than 0.05 lbs.

Significantly, the modulating elements 110 are also configured for modulating, and in particular, incrementally increasing, the tensile stiffness 250 of the tubular support structure 100 in response to axially stretching the tubular support structure 100, such that the primary tensile stiffness 250 of the tubular support structure 100 transitions to one or more higher secondary tensile stiffnesses 252, as illustrated in FIG. 21. In particular, in the same manner that the modulation of the bending stiffness of the tubular support structure 100 in response to laterally deflecting the tubular support structure 100 in a particular bending direction will create one or more inflection points 204 that define the transitions between the primary bending stiffness 200 and the secondary bending stiffness(s) 202, as illustrated in FIG. 20, the modulation of the tensile stiffness of the tubular support structure 100 in response to axially stretching the tubular support structure 100 will create one or more inflection points 254 that define the transitions between the primary tensile stiffness 250 within a primary axial stretch range and one or more secondary tensile stiffnesses 252, and in this case, two secondary tensile stiffnesses 252a, 252b, within a secondary axial stretch range that greater than the primary axial stretch range. At each inflection point 254, the tensile stiffness of the tubular support structure 100 increases. Thus, instead of continuing in an unmodulated manner (as illustrated by the dashed line), the primary tensile stiffness 250 of the tubular support structure 100 transitions to the secondary tensile stiffnesses 252, and in this case, to the tensile stiffness 252a, and then to the tensile stiffness 252b. Thus, after an initial axial stretch (during which the tubular support structure 100 has the primary tensile stiffness 250), the modulating elements 110 take up the tensile load in pure tension (during which the tubular support structure 100 has the secondary tensile stiffnesses 252) It should be appreciated that the primary tensile stiffness 250 of the tubular support structure 100 may transition to any number of secondary tensile stiffnesses 252, including only one secondary tensile stiffness 252 or more than two secondary tensile stiffnesses 252. Significantly, by increasing the tensile stiffness of the tubular support structure 100, the modulating elements 110 effectively increase the tensile strength of the tubular support structure 100 over that of a typical slotted tubular support structure having the same lateral bending stiffness as the primary bending stiffness 200 of the tubular support structure 100.

The tensile stiffness of the tubular support structure 100 may be increased from an initial tensile stiffness (in this case, the primary tensile stiffness 250) to a maximum tensile stiffness (in this case, the highest secondary tensile stiffness 252) by more than 50%, and preferably, by more than 100%, such that the tubular support structure 100 does not plastically deform under significant tensile forces. It should be appreciated that the tensile stiffness of the tubular support structure 100 is reversible in that, as the tubular support structure 100 is axially relaxed, the secondary tensile stiffness 252b will transition back to the secondary tensile stiffness 252a, which will then transition back to the primary tensile stiffness 250.

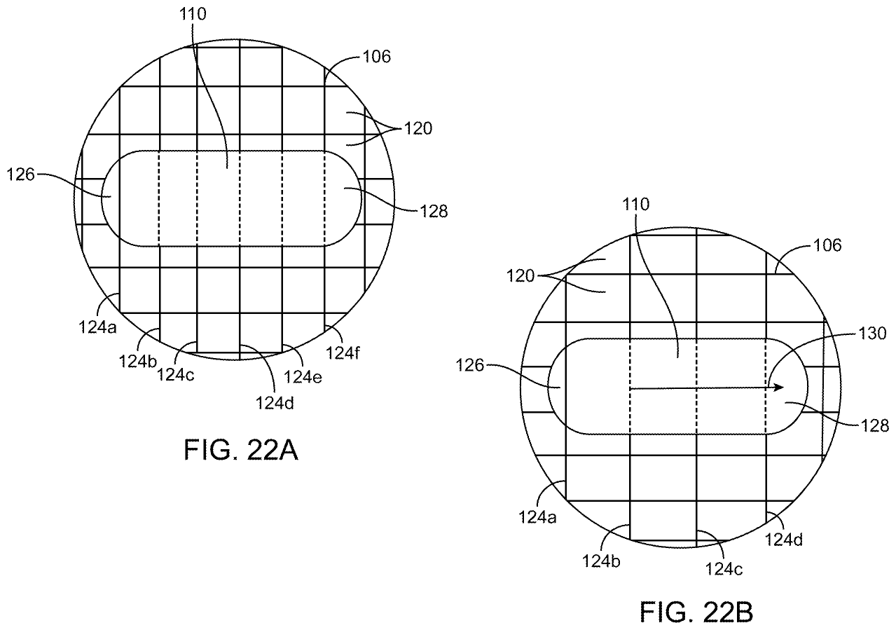

In one embodiment illustrated in FIGS. 22A and 22B, each modulating element 110 takes the form of a floating tab having a cantilevered end 126 affixed to the patterned frame structure 106 (and in particular, one or more of the members 124) and a free end 128 configured for translating relative to the patterned frame structure 106 (e.g., in the direction 129) as the patterned frame structure 106 is laterally deflected or axially stretched. In particular, as illustrated in FIG. 22A, the cantilevered end 126 of the floating tab 110 may be affixed to the member 124a of the patterned frame structure 106, while the free end 128 of the floating tab 110 floats relative to the members 124b-124f. As illustrated in FIG. 22B, as the tubular support structure 100 is laterally deflected or axially stretched, the spacings between the members 124 increases. The cantilevered end 126 of the floating tab 110 remains affixed to, and thus, translates with the member 124a, while the members 124b-124f translate relative to the free end 128 of the floating tab 110. As the patterned frame structure 106 is further laterally deflected or axially stretched, the free end 128 of the floating tab 110 will engage one or more of the members 124 (e.g., the members 124b-124d), at which point, the free end 128 of the floating tab 110 will translate with the members 124 with which the free end 128 of the floating tab 110 is engaged, thereby modulating the patterned frame structure 106, and in particular, increasing the bending stiffness and tensile stiffness of the tubular support structure 100. A particular floating tab 110 can be considered to be active or activated when its free end 116 engages the patterned frame structure 106, and can be considered to be inactive or deactivated when its free end 116 is translating relative to the patterned frame structure 106 or otherwise not engaged with the patterned frame structure 106. Further details discussing one embodiment of a patterned frame structure 106 and floating tabs 110 will be discussed in below.

The activation of the floating tabs 110 correspond to the inflection points 204 illustrated in FIG. 20. When the tubular support structure 100 is initially laterally deflected within the primary lateral deflection range, all of the floating tabs 110 are inactive, such that the bending stiffness of the tubular support structure 100 is not modulated, and will thus have a primary bending stiffness 200 within this primary lateral deflection range. However, when the tubular support structure 100 is subsequently laterally deflected within the secondary lateral deflection range, one or more of floating tabs 110 will be activated, such that the bending stiffness of the tubular support structure 100 will be modulated (i.e., increased), and will thus have one or more secondary bending stiffnesses 202 within the secondary lateral deflection range that are greater than the primary bending stiffness 200.

If the tubular support structure 100 only has one secondary bending stiffness 202 for each bending direction, all of the floating tabs 110 responsible for modulating the bending stiffness of the tubular support structure 100 for the respective bending direction (e.g., one of the modulating element columns 110a-110d) will be activated when the tubular support structure 100 is laterally deflected from the primary lateral deflection range to the secondary lateral deflection range. However, if the tubular support structure 100 has multiple secondary bending stiffnesses 202 for each bending direction, different sets of the floating tabs 110 responsible for modulating the bending stiffness of the tubular support structure 100 (e.g., two sets of floating tabs 110 within one of the modulating element columns 110a-110d) will be incrementally activated as the bending stiffness of the tubular support structure 100 is laterally deflected within the secondary lateral deflection range. That is, a first set of floating tabs 110 will be activated when the bending stiffness of the tubular support structure 100 is laterally deflected at a first magnitude within the secondary lateral deflection range, then a second set of floating tabs 110 are activated (while the first set of floating tabs 110 remain active) when the bending stiffness of the tubular support structure 100 is further laterally deflected at a second higher magnitude within the secondary lateral deflection range.

It should be appreciated that when the tubular support structure 100 is laterally deflected from the secondary lateral deflection range back to the primary lateral deflection range, all of the floating tabs 110 will be deactivated, such that the bending stiffness of the tubular support structure 100 is not modulated, and will thus again have a primary bending stiffness 200 within this primary lateral deflection range.

The activation of the floating tabs 110 also correspond to the inflection points 254 illustrated in FIG. 21. When the tubular support structure 100 is initially axially stretched within the primary lateral deflection range, all of the floating tabs 110 are inactive, such that the tensile stiffness of the tubular support structure 100 is not modulated, and will thus have a primary tensile stiffness 250 within this primary lateral deflection range. However, when the tubular support structure 100 is subsequently axially stretched within the secondary axial stretch range, one or more of floating tabs 110 will be activated, such that the tensile stiffness of the tubular support structure 100 will be modulated (i.e., increased), and will thus have one or more secondary tensile stiffnesses 252 within the secondary axial stretch range that are greater than the primary tensile stiffness 250.

If the tubular support structure 100 only has one secondary tensile stiffness 252, all of the floating tabs 110 will be activated when the tubular support structure 100 is axially stretched from the primary axial stretch range to the secondary axial stretch range. However, if the tubular support structure 100 has multiple secondary tensile stiffnesses 252, different sets of the floating tabs 110 (e.g., one or more floating tabs 110 within each of the modulating element columns 110a-110d that form circumferentially disposed sets of floating tabs 110) will be incrementally activated as the tubular support structure 100 is axially stretched within the secondary axial stretch range. That is, a first set of floating tabs 110 (e.g., one set of circumferentially disposed floating tabs 110) will be activated when the tubular support structure 100 is axially stretched at a first magnitude within the secondary axial stretch range, then a second set of floating tabs 110 are activated (while the first set of floating tabs 110 remain active) when the tubular support structure 100 is further axially stretched at a second higher magnitude within the secondary axial stretch range.

It should be appreciated that when the tubular support structure 100 axially relaxes from the secondary axial stretch range back to the primary axial stretch range, all of the floating tabs 110 will be deactivated, such that the tensile stiffness of the tubular support structure 100 is not modulated, and will thus again have a primary tensile stiffness 250 within this primary axial stretch range. Notably, in the illustrated embodiment, the activation sequence of the floating tabs 110 to modulate the tensile stiffness of the tubular support structure 100 will be the same as the activation sequence of the floating tabs 110 to modulate the bending stiffness of the tubular support structure 100. As such, the number of inflection points 254 and secondary tensile stiffnesses 252 will track the number of inflection points 204 and secondary tensile stiffnesses 202 of the patterned frame structure 106.

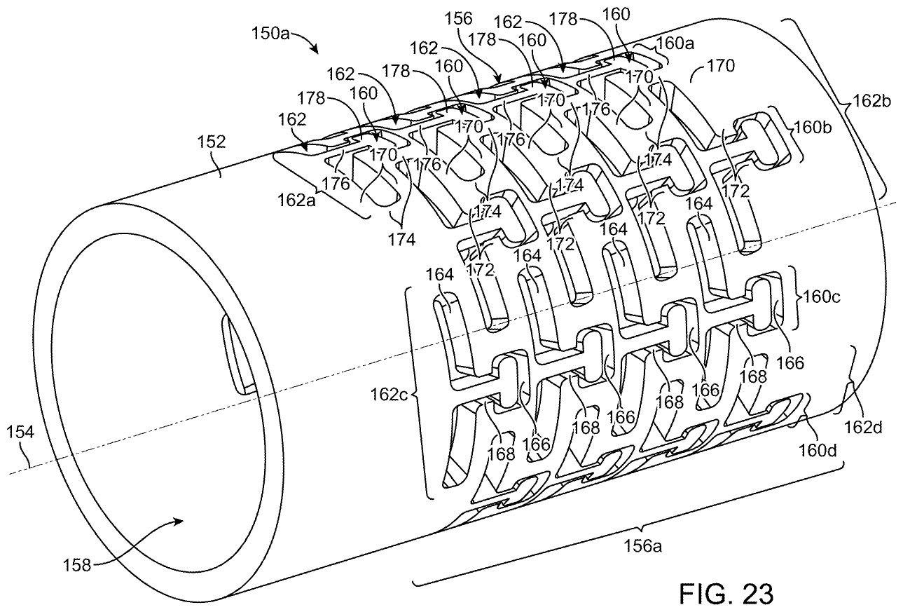

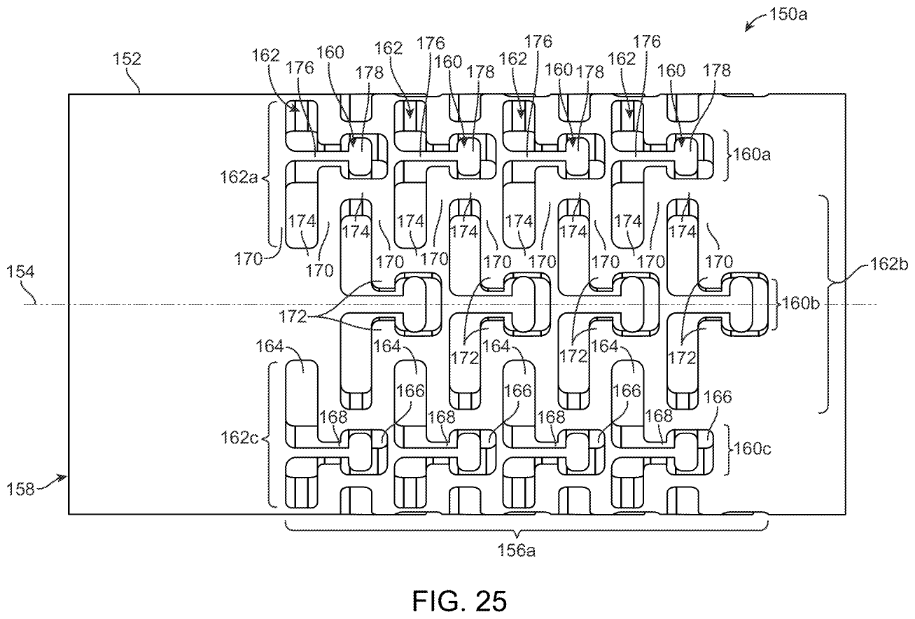

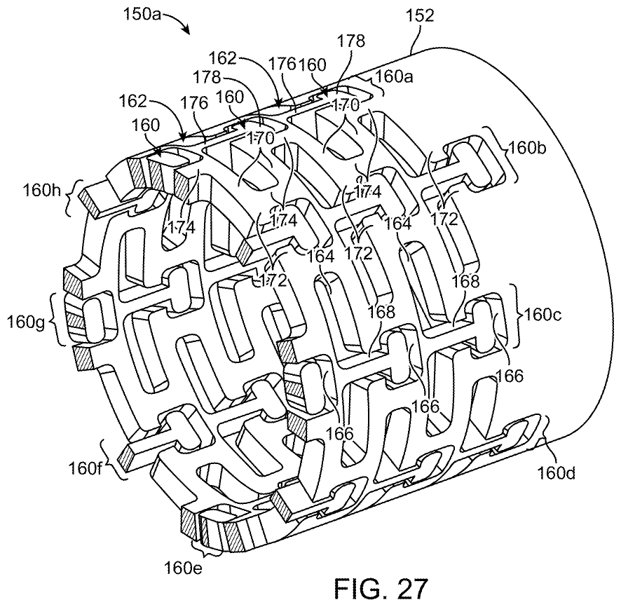

Referring now to FIGS. 23-27, one specific embodiment of a tubular support structure 150a will be described. Although the tubular support structure 150a is shown to have a relatively short length for purposes of illustration, it should be appreciated that the tubular support structure 150a may have any suitable length, including extending the entire length of the catheter or guidewire.

The tubular support structure 150a generally comprises an elongate tubular body 152 having a longitudinal axis 154, a patterned frame structure 156a formed into the tubular body 152, an inner lumen 158 axially disposed along the longitudinal axis 154 of tubular body 152, and a plurality of mechanical property modulating elements 160 (in this case, floating tabs 160) axially spaced along and circumferentially spaced around the patterned frame structure 156.

The patterned frame structure 156, along with the floating tabs 160, may be formed into the tubular body 152 using any suitable process, including laser cutting, etching, waterjet cutting, electrical discharge machining, grinding, milling, casting, molding, among other methods. Although the patterned frame structure 156a is illustrated as extending along substantially the entire length and around the entire circumference of the tubular body 152, it should be appreciated that the patterned frame structure 156a may extend along a lengthwise portion of the tubular body 152, including only the proximal portion of the tubular body 152 or only the distal portion of the tubular body 152, or around a circumferential portion of the tubular body 152, e.g., only 180° or 90° (e.g., in the case where it is desirable for the patterned frame structure 156a to have a radially anisotropic primary bending stiffness).

The patterned frame structure 156a has a plurality of bending flexibility enhancement apertures 162 configured for decreasing the bending stiffness of the patterned frame structure 156a to create a primary bending stiffness in at least one bending direction. In the illustrated embodiment, the apertures 162 are formed entirely through the tubular body 152, such that the inner lumen 158 of the tubular support structure 150a is exposed through the apertures 162, although in alternative embodiments, the apertures 162 are formed partially into the tubular body 152, so that the inner lumen 158 of the tubular support structure 150a is not exposed through the apertures 162.

In the illustrated embodiment, the apertures 162 are arranged as sets 162a-162h, and in this case circumferentially aligned sets (i.e., in columns) that are circumferentially spaced from each other around the patterned frame structure 156a in a uniform manner, with the apertures 162 of each of the columns 162a-162h being axially spaced apart from each other along the patterned frame structure 156a in a uniform manner. In the illustrated embodiment, the apertures 162 are arranged in eight columns 162a-162h circumferentially spaced apart from each other by 45°, and each of the aperture columns 162a-162h has four apertures 162. It should be appreciated that the apertures 162 may be arranged in any suitable number of columns, and each of the aperture columns 162a-162h may have any suitable number of apertures 162.

In the illustrated embodiment, the tubular support structure 150a has a radially isotropic primary bending stiffness, although in alternative embodiments, the tubular support structure 150a may have a radially anisotropic primary bending stiffness. For example, in an alternative embodiment, the tubular support structure 150a may have only two diametrically opposing aperture columns (e.g., aperture columns 162a, 162e), such that the magnitudes of the primary bending stiffness of the patterned frame structure 156a are equal when laterally deflected in bending directions towards either of the aperture columns 162a, 162e, which are increased when laterally deflected at 90° from the opposing aperture columns 162. As another example, patterned frame structure 156a may have a single aperture column (e.g., aperture column 162c), such that the magnitude of the primary bending stiffness of the tubular support structure 150a when laterally deflected in a bending direction towards the single aperture column 162c is less than the magnitude of the primary bending stiffness of the tubular support structure 150a when laterally deflected in a bending direction away from the single aperture column 162c. In other embodiments, the patterned frame structure 156a may have the same number of aperture columns 162 as shown in FIG. 23, but some of the aperture columns 162 may lack the floating tabs 160, the stem element 172, or the extensions 172, so that stiffness is not changed by those elements interacting when the tubular support structure 150a is bent laterally.

In the illustrated embodiment, each aperture 162 has a transverse slot 164, a retainer opening 166, and an axial channel 168. The transverse slot 164 is coextensive with the retainer opening 166, and in particular, the axial channel 168 connects the transverse slot 164 and retainer opening 166 together. Although the slots 164 are illustrated as being generally rectangular, the slots 164 may have other elongated shapes, such as elliptical or oval. Furthermore, in alternative embodiments, rather than slots 164, slits (not shown) may be formed within the tubular body 152. In the illustrated embodiment, each retainer opening 166 is rectangular in shape, but in alternative embodiments, the retainer opening 166 may have a different geometric shape. However, it is preferable that the retainer opening 166 be shaped to facilitate axial translation of a floating tab 160 therein, as will be described in further detail below.

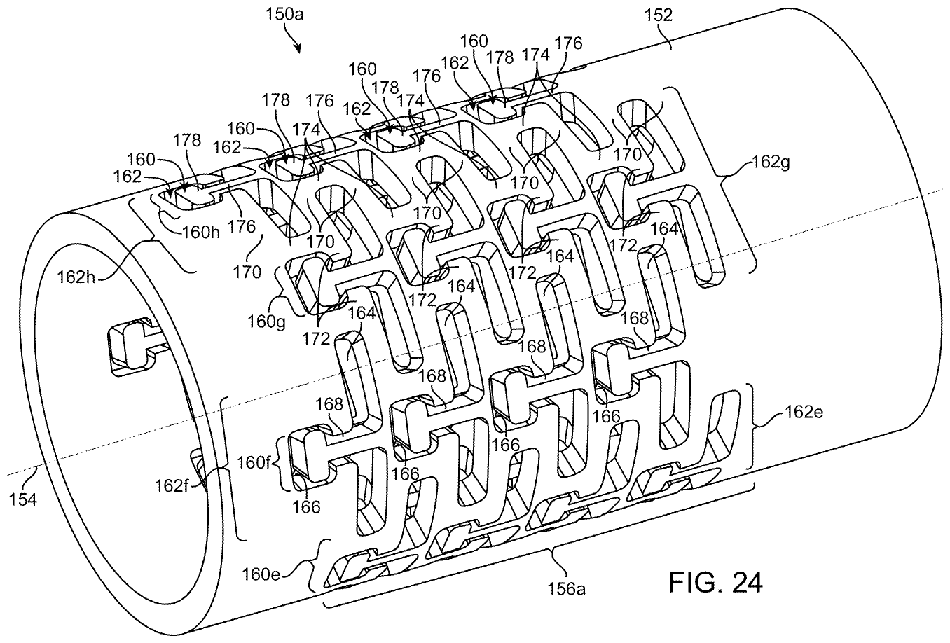

In the illustrated embodiment, each of the aperture columns 162a-162h is axially offset from the two most adjacent aperture columns, such that the transverse slots 164 of the respective aperture column interleave with the transverse slots 164 of the aperture columns on both sides of the respective aperture column 162a-162h (e.g., the transverse slots 164 of the aperture column 162b interleaves with the transverse slots 164 of the aperture columns 162a, 162c illustrated in FIG. 23; or transverse slots 164 of the aperture column 162g interleaves with the transverse slots 164 of the aperture columns 162f, 162h illustrated in FIG. 24). Furthermore, the transverse slots 164 of each of the aperture columns 162a-162h is circumferentially aligned with the retainer openings 166 of the two most adjacent aperture columns (e.g., the transverse slots 164 of the aperture column 162b are circumferentially aligned with the retainer openings 166 of the aperture columns 162a, 162c illustrated in FIG. 23; or the transverse slots 164 of the aperture column 162g are circumferentially aligned with the retainer openings 166 of the aperture columns 162f, 162h illustrated in FIG. 24).

Substantially transverse members 170 are formed between the interleaved transverse slots 164 of adjacent apertures columns 162a-162h, such that circumferentially aligned sets (i.e., in columns) of transverse members 170 are circumferentially spaced around the patterned frame structure 156, with the transverse members 170 of each of the columns of transverse members 170 being axially spaced apart from each other along the patterned frame structure 156. Each transverse member 170 extends between the retainer opening 166 and axial channel 168 of the two most adjacent aperture columns, with the end of the transverse member 170 adjacent the respective axial channel 168 forming an extension 172 that impinges on the respective axial channel 168. As will be described in further detail below, each floating tab 160 is mechanically coupled to at least one of the transverse members 170 (and in this case, a pair of circumferentially aligned transverse members 170), such that the floating tab 160 axially translates as the transverse members 170 flex in response to laterally deflecting or axially stretching the patterned frame structure 156. The extension 172 serves as an abutment to the floating tab 160 associated with the retainer opening 166 that communicates with the respective axial channel 168. In particular, each pair of extensions 172 of two adjacent circumferentially aligned transverse members 170 defines an axial channel 168, and serves as an abutment for the floating tab 160 associated with the retainer opening 166 in communication with that respective axial channel 168. The pair of extensions 172 are configured for laterally flexing as the floating tab 160 engages them, thereby tempering the secondary bending stiffness of the tubular support structure 150a.

Axial connecting members 174 rigidly couple the transverse members 170 of each column of transverse members 170 together. In particular, each connector member 174 rigidly couples the ends of two axially adjacent transverse members 170 together in an alternating fashion, such that the transverse members 170 and connecting members 174 of each column form a zig-zag pattern that axially extends along the patterned frame structure 156.

Although the transverse slots 164 and transverse members 170 are illustrated as being perfectly transverse in that they extend perpendicularly to the longitudinal axis 154 of the tubular body 152, the transverse slots 164 and transverse members 170 may not be perfectly transverse. For example, the slots 164 and/or members 170 may be substantially transverse in that they may extend within an angular range of ±10° from the perpendicular to the longitudinal axis 154 of the tubular body 152. Furthermore, although the channels 168 and connecting members 174 are illustrated as axially extending along the patterned frame structure 156a in that they extend parallel to the longitudinal axis 154 of the tubular body 152, the channels 168 and connecting members 174 may not be perfectly axial. For example, the channels 168 and connecting members 174 may be substantially axial in that they may extend within an angular range of ±10° from the longitudinal axis 154 of the tubular body 152. It should also be appreciated that the slots 164, transverse members 170, channels 168, and/or connecting members 174 may be disposed obliquely at an angle relative to the longitudinal axis 154 of the tubular body 152, e.g., at 45° to the longitudinal axis 154 of the tubular body 152.

In the illustrated embodiment, the apertures 162 maintain the same geometry through the entire thickness of the tubular body 152, although in alternative embodiments, the geometry of the apertures 162 may change as they traverse through the thickness of the tubular body 152. Furthermore, it should be appreciated that numerous other geometries for the apertures 162 are contemplated, including circles and polygons, such as triangles, squares, rectangles, parallelograms, rhombuses, trapezoids, etc. While the apertures 162 are disposed in the patterned frame structure 156, such that they do not overlap with each other, alternatively, at least some of the apertures 162 may overlap each other.

Although the patterned frame structure 156a is illustrated as being regular (i.e., a pattern that predictably repeats), the patterned frame structure 156a may be irregular (i.e., a pattern that unpredictably repeats). Furthermore, although the slots 164 and members 170, 174 are illustrated as being of equal size, the size of the slots 164 and members 170, 174 may vary between each other. Furthermore, although the patterned frame structure 156a is uniform along the length of the tubular body 152, the size, shape, and/or angle of the apertures 162 and members 170, 174 may be varied along the length of the tubular body 152 in order to vary the primary bending stiffness of the patterned frame structure 156a along the length of the tubular body 152.

The floating tabs 160 modulate (by increasing) the bending stiffness and tensile stiffness of the patterned frame structure 156. In the illustrated embodiment, the floating tabs 160 are integrally formed with the patterned frame structure 156. Like the modulating elements 110 illustrated in FIGS. 7-10, the floating tabs 160 in the embodiment illustrated in FIGS. 23-27 are arranged as sets 160a-160h, and in this case circumferentially aligned sets (i.e., in columns) that are circumferentially spaced from each other around the patterned frame structure 156a in a uniform manner, with the floating tabs 160 of each of the columns 160a-160h being axially spaced apart along the patterned frame structure 156a in a uniform manner. In the illustrated embodiment, the floating tabs 160 are arranged in eight columns 160a-160h circumferentially spaced apart from each other by 45°, and each of the floating tab columns 160a-160h has four floating tabs 160. Thus, the bending stiffness of the tubular support structure 150a may be modulated in eight different radial directions spaced apart by 45°. It should be appreciated that the floating tabs 160 may be arranged in any suitable number of columns, and each of the floating tab columns 160a-160h may have any suitable number of floating tabs 160.

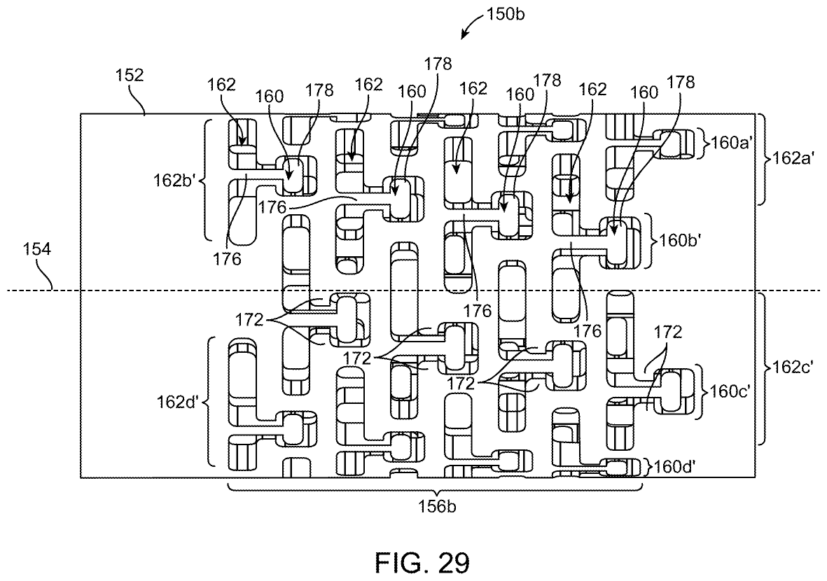

In this embodiment, the floating tab columns 160a-160h are identical, and thus, the floating tabs 160 are configured for increasing the bending stiffness of the tubular support structure 150a from an initial radially isotropic primary bending stiffness to one or more radially isotropic secondary bending stiffnesses. In an alternative embodiment, the floating tabs 160 may be configured for increasing the bending stiffness of the tubular support structure 150a from an initial radially isotropic primary bending stiffness to one or more radially anisotropic secondary bending stiffnesses. For example, the floating tab columns 160a-160h may not be identical, or the tubular support structure 150a may have only two diametrically opposing columns of floating tabs 160 (e.g., similar to the arrangement of modulating elements 110 illustrated in FIGS. 12-13) disposed on the patterned frame structure 156, or a single column of floating tabs 160 (e.g., similar to the arrangement of modulating elements 110 illustrated in FIGS. 15-16) disposed on the patterned frame structure 156. Although the floating tabs 160 are illustrated in FIGS. 23-27 as being arranged in circumferentially aligned sets, an alternative embodiment of a tubular support structure 150b comprises a patterned frame structure 156b having floating tabs 160 that, similar to the arrangement of modulating elements 110 illustrated in FIG. 12, may be are arranged in eight circumferentially offsetcircumferentially misaligned sets (only sets 160a′-160d′ shown), as well as associated apertures 162 that are arranged in eight circumferentially offsetcircumferentially misaligned sets (only sets 162a′-162d′ shown), as illustrated in FIG. 29.

It should be appreciated that the floating tabs 160 may be arranged in any suitable number of columns, and each of the floating tab columns may have any suitable number of apertures floating tabs 160.

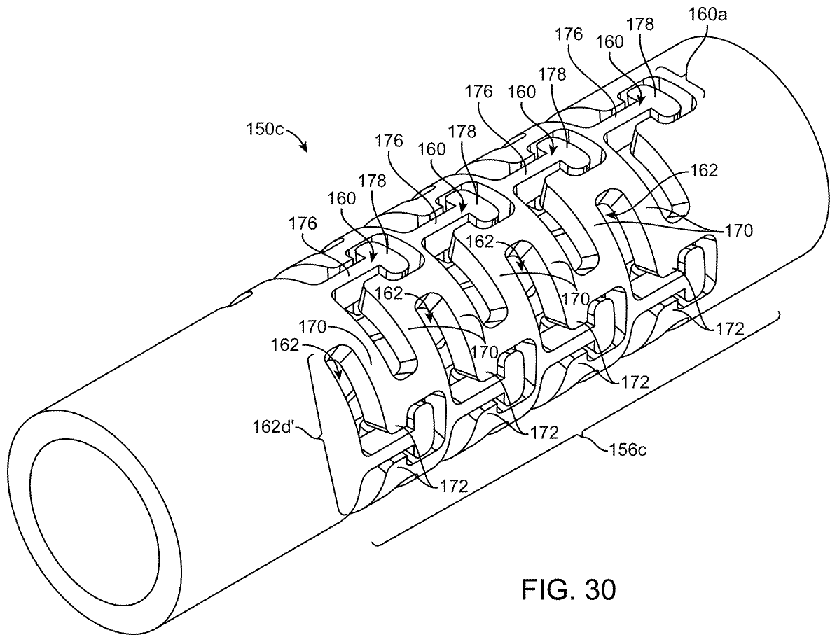

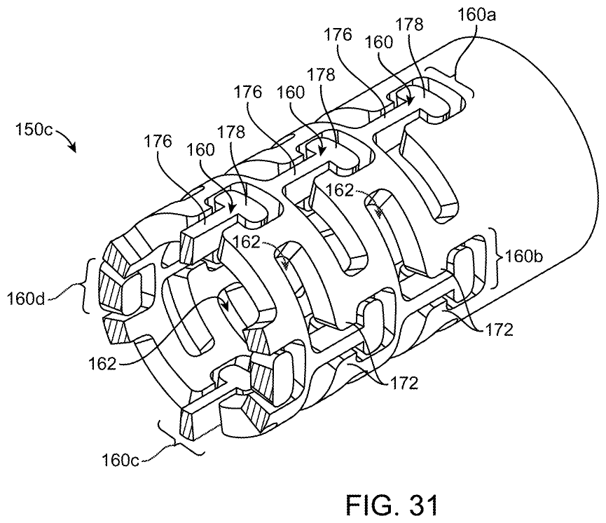

For example, an alternative embodiment of a tubular support structure 150c illustrated in FIGS. 30-31, comprises a patterned frame structure 156c having floating tabs 160 arranged in four circumferentially aligned sets (columns) 160a-160d, as well as associated apertures 162 that are arranged in four circumferentially aligned sets (columns). Notably, the diameter of the tubular support structure 150c is smaller than the diameter of the tubular support structure 150a illustrated in FIGS. 23-27, such that the four columns of apertures 162 are sufficient for providing a radially isotropic primary bending stiffness to the tubular support structure 150c, while the four columns of floating tabs are sufficient for providing a radially isotropic secondary bending stiffness to the tubular support structure 150c.

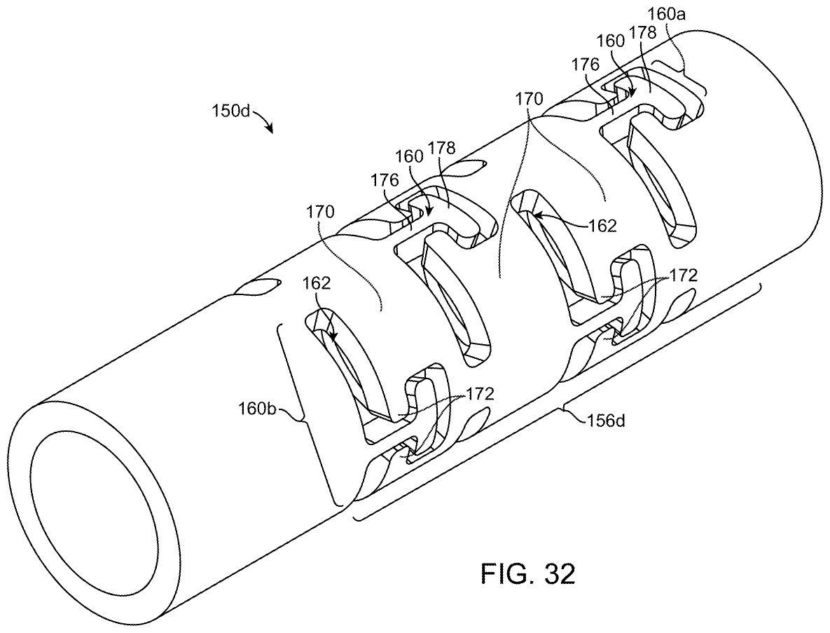

As another example, an alternative embodiment of a tubular support structure 150d illustrated in FIG. 32, is similar to the tubular support structure 150c illustrated in FIGS. 30-31, with the exception that each of the floating tab columns has only two floating tabs 160, and each of the associated columns of apertures has only two apertures 162. As a result, the width of each of the resulting transverse members 170 of the tubular support structure 150d is greater than the width of each of the transverse members 170 of the tubular support structure 150c illustrated in FIGS. 30-31, such that the primary bending stiffness of the tubular support structure 150d will be greater than the primary bending stiffness of the tubular support structure 150c.

Referring back to FIGS. 23-27, each of the floating tabs 160 comprises a cantilevered end in the form of a stem element 176 and a free end in the form of an enlarged element 178. In the illustrated embodiment, each of the floating tabs 160 is T-shaped, with the stem element 176 forming the base of the “T,” and enlarged element 178 forming the cross of the “T.” In the illustrated embodiment, the opposing ends of the enlarged element 178 are generally rectilinear, although in alternative embodiments, the opposing ends of the enlarged element 178 may be rounded. The stem element 176 resides within the respective axial channel 168, and axially extends from a respective pair of circumferentially aligned transverse members 170, across the respective transverse slot 164, and into the respective retainer opening 166. Thus, stem element 176 remains affixed to, and thus, translates with the respective transverse member 170.

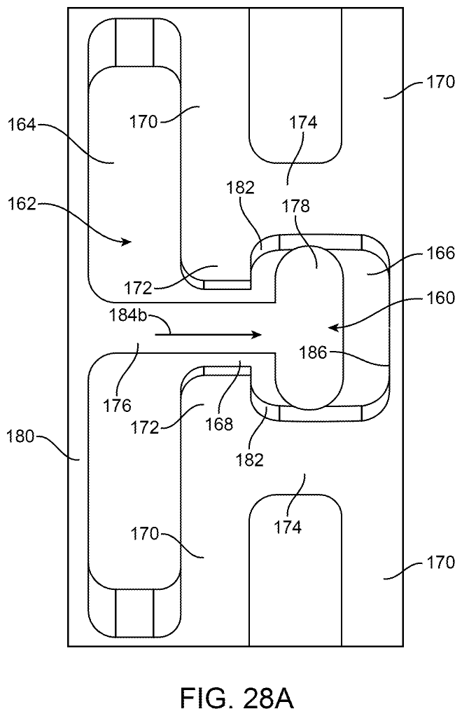

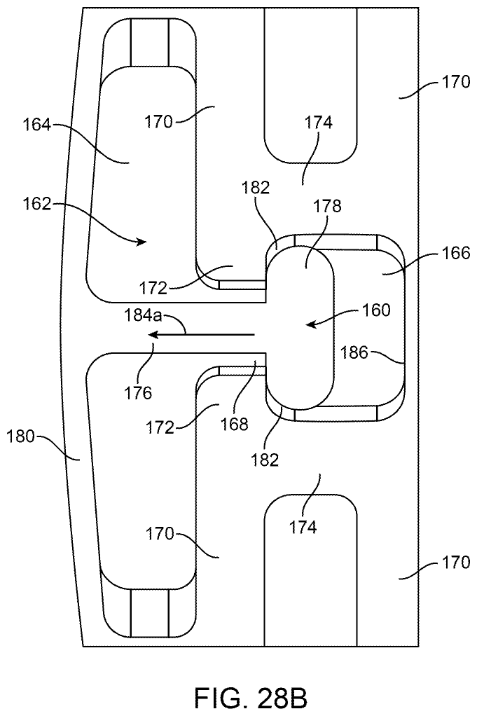

Referring specifically to FIGS. 28A-28B, the stem element 176 is mechanically coupled between the respective pair of circumferentially aligned transverse members 170 via a bridge member 180. The enlarged elements 178 are geometrically similar to, but smaller in size than, the retainer openings 166, such that the enlarged elements 178 are free to axially translate within the retainer openings 166 to alternately engage or disengage an abutment edge 182 of the respective retainer opening 166 (and in particular, the pair of extensions 172 of the transverse members 170 associated with the two most adjacent aperture columns relative to the aperture column associated with the respective transverse member 170).

Thus, each enlarged element 178 of each floating tab 160 is configured for axially translating in one axial direction 184a within the respective retainer opening 166, thereby engaging the abutment edge 182 of the respective retainer opening 166 (as best shown in FIG. 28B), when the patterned frame structure 156a is laterally deflected in a bending direction toward the floating tab 160 or axially stretched. At this point, the respective floating tab 160 can be considered to be active or activated. Notably, each bridge member 180 to which the stem element 176 of a respective floating tab 160 is affixed flexes to axially translate the enlarged element 178 of the respective floating tab 160 in the axial direction 184a in engagement with the abutment edge 182 of the respective retainer opening 166.

In contrast, enlarged element 178 of each floating tab 160 is configured for axially translating in an opposite axial direction 184b within the respective retainer opening 166, thereby disengaging the abutment edge 182 of the respective retainer opening 166 (as best shown in FIG. 28A), when the patterned frame structure 156a is straightened and/or axially relaxed. At this point, the respective floating tab 160 can be considered to be inactive or deactivated. Notably, each bridge member 180 to which the stem element 176 of a respective floating tab 160 is affixed relaxes to axially translate the enlarged element 178 of the respective floating tab 160 in the axial direction 184b in disengagement with the abutment edge 182 of the respective retainer opening 166.

It should be appreciated that, although the retainer openings 166 and associated floating tabs 160 are all axially oriented in the same direction, such that all of the enlarged elements 178 are configured for axially translating in the same axial direction 184a to engage the abutment edges 182 of the retainer openings 166 when the patterned frame structure 156a is laterally deflected or axially stretched, and all of the enlarged elements 178 are configured for axially translating in the same axial direction 184b to disengage the abutment edges 182 of the retainer openings 166 when the patterned frame structure 156a is straightened and/or axially relaxed, some of the retainer openings 166 and associated floating tabs 160 may be axially oriented opposite to others of the associated retainer openings 166 and associated floating tabs 160, such a first set of enlarged elements 178 are configured for axially translating in the axial direction 184a, and a second set of the enlarged elements 178 are configured for axially translating in the axial direction 184b, engage the abutment edges 182 of the retainer openings 166 when the patterned frame structure 156a is laterally deflected or axially stretched, and the first set of enlarged elements 176 are configured for axially translating in the axial direction 184b, and the second set of the enlarged elements 178 are configured for axially translating in the axial direction 184a, to disengage the abutment edges 182 of the retainer openings 166 when the patterned frame structure 156a is straightened and/or axially relaxed.

It should be appreciated that, as with the floating tabs 110 illustrated in FIGS. 7-10, the activation of the floating tabs 160 may correspond to inflection points 204, 254 in the bending stiffness and tensile stiffness of the tubular support structure 150a illustrated in FIGS. 20-21. Thus, when the tubular support structure 150a is initially laterally deflected within the primary lateral deflection range or axially stretched within the primary axial stretch range, all of the floating tabs 160 are inactive, such that the bending stiffness or tensile stiffness of the tubular support structure 150a is not modulated, and will thus have a primary bending stiffness 200 within this primary lateral deflection range (shown in FIG. 20) or primary tensile stiffness 250 within this primary axial stretch range (shown in FIG. 21). However, when the tubular support structure 150a is subsequently laterally deflected within the secondary lateral deflection range or axially stretched within the secondary axial stretch range, one or more of the floating tabs 160 will be activated, such that the bending stiffness or tensile stiffness of the tubular support structure 150a will be modulated (i.e., increased), and will thus have one or more secondary bending stiffnesses 202 within the secondary lateral deflection range that are greater than the primary bending stiffness 200 (shown in FIG. 20) or one or more secondary tensile stiffnesses 252 within the secondary lateral deflection range that are greater than the primary tensile stiffness 250 (shown in FIG. 21).

Notably, the lengths of the floating tabs 160, and in particular, the lengths of the stem elements 176 of the floating tabs 160, may be designed to select the locations and number of inflection points 204 of the bending stiffness and inflection points 254 of the tensile stiffness of the tubular support structure 150a. In particular, the length of each stem element 176 defines the clearance between the enlarged element 178 of the respective floating tab 160 and the abutment edge 182 of the respective retainer opening 166, thereby defining the location of the inflection points 204 of the bending stiffness and inflection points 254 of the tensile stiffness of the tubular support structure 150a. That is, as the length of a particular stem element 176 is increased, the clearance between the enlarged element 178 of the respective floating tab 160 and the abutment edge 182 of the respective retainer opening 166 correspondingly increases, thereby increasing the magnitude of the lateral deflection at which a particular inflection point 204 occurs (i.e., moving the inflection point 204 to the right on the x-axis illustrated in FIG. 20) or increasing the magnitude of the axial stretch at which a particular inflection point 254 occurs (i.e., moving the inflection point 254 to the right on the x-axis illustrated in FIG. 21). In contrast, as the length of a particular stem element 176 is decreased, the clearance between the enlarged element 178 of the respective floating tab 160 and the abutment edge 182 of the respective retainer opening 166 correspondingly decreases, thereby decreasing the magnitude of the lateral deflection at which a particular inflection point 204 occurs (i.e., moving the inflection point 204 to the left on the x-axis illustrated in FIG. 20) or decreasing the magnitude of the axial stretch at which a particular inflection point 254 occurs (i.e., moving the inflection point 254 to the left on the x-axis illustrated in FIG. 21).

The number of inflection points 204 of the bending stiffness of a tubular support structure, and correspondingly, the number of inflection points 254 of the tensile stiffness of the tubular support structure, can be selected by accordingly varying the lengths between the stem elements 176 of different sets of floating tabs 160.

For example, the lengths of the stem elements 176 of all of the floating tabs 160 in the tubular support structures 150a-150d illustrated in FIGS. 23-32 are uniform, such that all of the floating tabs 160 engage the abutment edges 182 of the respective patterned frame structures 156a-156d (shown in FIGS. 28A-28B) (and thus, all activate) at the same time. As a result, the bending stiffness of each of the tubular support structures 150a-150d will have a single inflection point 204a that defines the boundary between the primary bending stiffness 200 and the secondary bending stiffness 202, and a single inflection point 254a that defines the boundary between the primary tensile stiffness 250 and the secondary tensile stiffness 252.

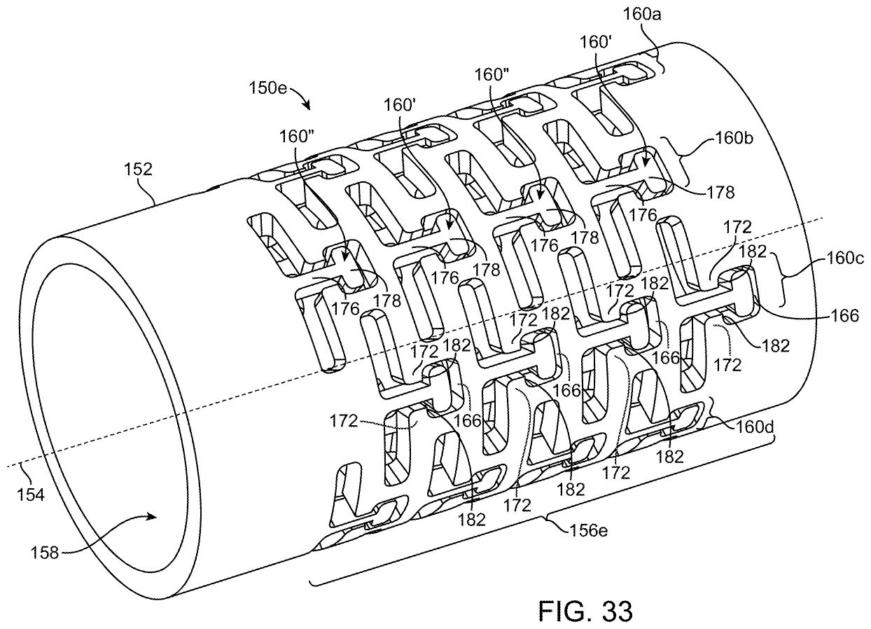

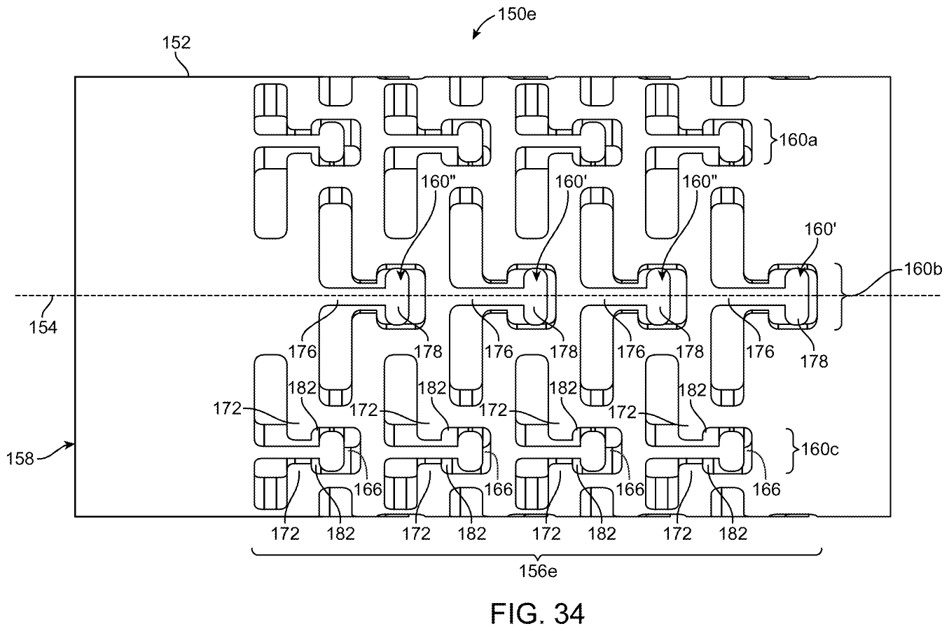

In contrast, the lengths of the stem elements 176 of the floating tabs 160 in the tubular support structure 150e illustrated in FIGS. 33-34 are non-uniform. In particular, in each of the floating tab columns (only columns 160a-160d shown), the lengths of the stem elements 176 of a first set of floating tabs 160′ are longer than the stem elements 176 of a second different set of floating tabs 160′, such that the two sets of floating tabs 160 engage the abutment edges 182 of the patterned frame structure 156e (and thus, activate) at two different times. As a result, the bending stiffness of the tubular support structure 150e will have two inflection points 204a, 204b, one inflection point 204a that defines the boundary between the primary bending stiffness 200 and the secondary bending stiffness 202a, and a second inflection point 204b that defines the boundary between the two secondary bending stiffnesses 202a, 202b, as illustrated in FIG. 20; and the tensile stiffness of the tubular support structure 150e will have two inflection points 254a, 254b, one inflection point 254a that defines the boundary between the primary tensile stiffness 250 and the secondary tensile stiffness 252a, and a second inflection point 254b that defines the boundary between the two secondary tensile stiffnesses 252a, 252b, as illustrated in FIG. 21.

It should be appreciated that, although the first and second sets of floating tabs 160′, 160″ alternate and are equal in number for each floating tab columns, in alternative embodiments, the number of the floating tabs in the respective first and second sets of floating tabs 160′, 160″ may be different (e.g., one floating tab may be provided for the first set of floating tabs 160′, and three floating tabs may be provided for the second set of floating tabs 160″) or a different ordering or sequence of the first and second sets of floating tabs 160′, 160″ may be provided (e.g., two immediately adjacent floating tabs 160′ may be provided on one end of a floating tab column, and two immediately adjacent floating tabs 160″ may be provided on the other end of the floating tab column).

It should be appreciated the lengths of stem elements 176 of more than two sets of floating tabs 160 may be different from each other, such that the sets of floating tabs 160 engage the abutment edges 182 (and thus, activate) of the retainer openings 166 at more than two different times. As a result, the bending stiffness of tubular support structure will have more than two inflection points 202, one inflection point 204a that defines the boundary between the primary bending stiffness 200 and the secondary bending stiffness 202a, and at least two inflection points 204b, etc., that define the boundaries between at least three secondary bending stiffnesses 202a, 202b, etc.; and the tensile stiffness of the tubular support structure will have more than two inflection points 252, one inflection point 254a that defines the boundary between the primary tensile stiffness 250 and the secondary tensile stiffness 252a, and at least two inflection points 254b, etc., that define the boundaries between at least three secondary tensile stiffnesses 202a, 202b, etc.

Notably, the lengths of the pairs of extensions 172 may be designed to select the magnitudes of the secondary bending stiffnesses 202a, 202b or secondary bending stiffness 202a (illustrated in FIG. 20) or secondary tensile stiffnesses 252a, 252b or tensile stiffness 252a (illustrated in FIG. 21). In particular, the lengths of each pair of extensions 172 define the flexibility of the abutment edge 182 of the respective retainer opening 166. That is, as the lengths of a particular pair of extensions 172 increases, the flexibility of the abutment edge 182 of the respective retainer opening 166 increases, thereby decreasing the magnitude of the secondary bending stiffness 202a (profiles 202a, 202b) or the secondary tensile stiffness 252a (or profiles 252a, 252b). In contrast, as the lengths of a particular pair of extensions 172 decreases, the flexibility of the abutment edge 182 of the respective retainer opening 166 decreases, thereby increasing the magnitude of the secondary bending stiffness 202a (or profiles 202a, 202b) or the secondary tensile stiffness 252a (or profiles 252a, 252b).

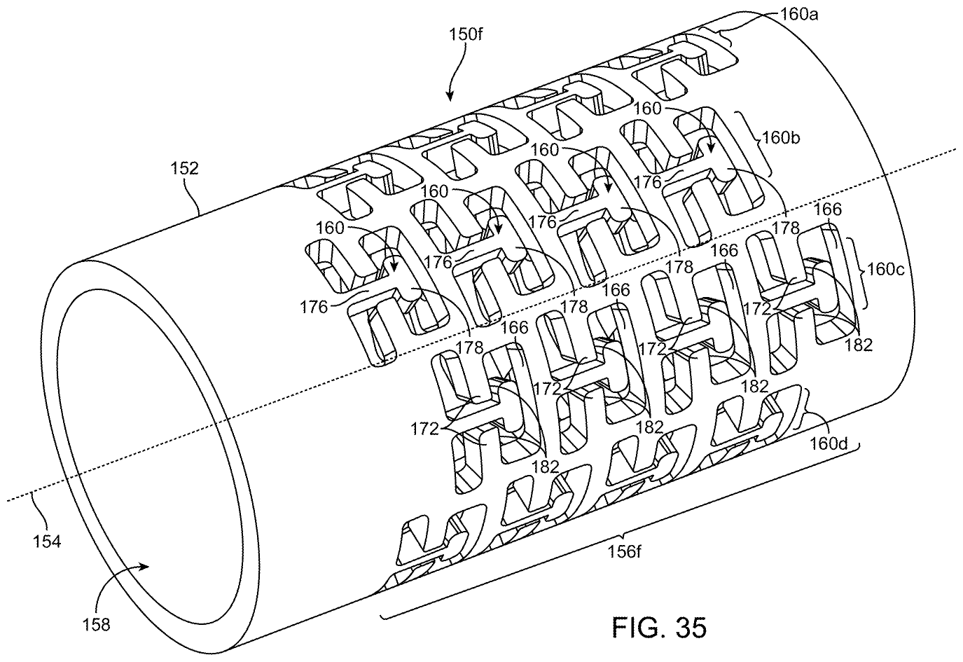

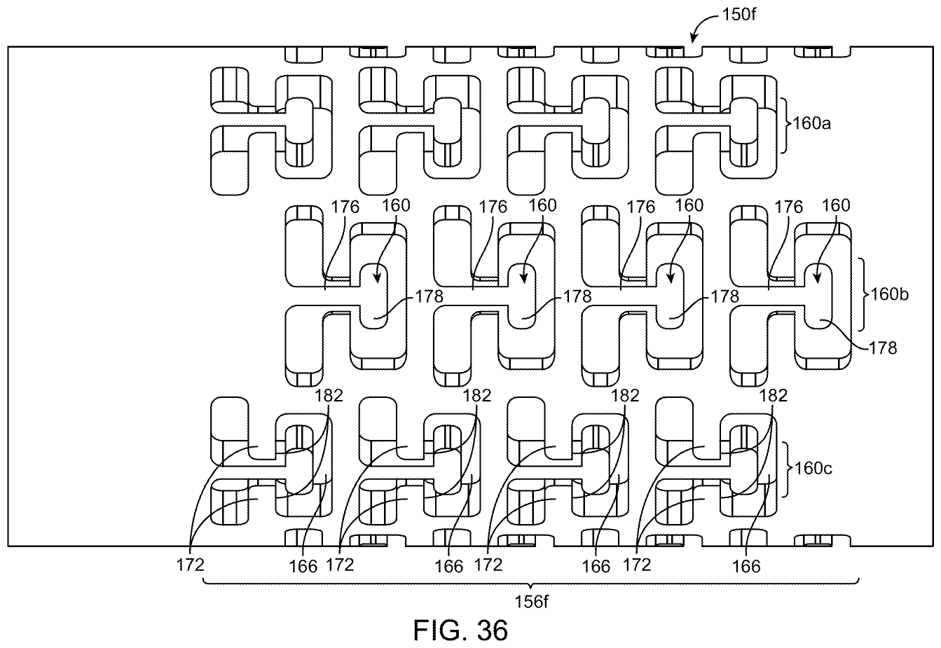

For example, the lengths of the pairs of extensions 172 in the tubular support structures 150a-150e illustrated in FIGS. 23-34 are relatively short. As such, the flexibility of the abutment edge 182 of each of the retainer openings 166 of the patterned frame structures 156a-156e will be relatively low, and thus, the magnitude of the secondary bending stiffness 202a (profiles 202a, 202b) or the secondary tensile stiffness 252a (or profiles 252a, 252b) of the tubular support structures 150a-150e will be relatively high. In contrast, the lengths of the pairs of extensions 172 in the tubular support structure 150f illustrated in FIGS. 35-36 are relatively long. As such, the flexibility of the abutment edge 182 of each of the retainer openings 166 of the patterned frame structure 156f will be relatively high, and thus, the magnitude of the secondary bending stiffness 202a (profiles 202a, 202b) or the secondary tensile stiffness 252a (or profiles 252a, 252b) of the tubular support structure 150f will be relatively low. It should be noted that the transverse dimensions of the retainer openings 166 in the patterned frame structure 156f of FIGS. 35-36 are larger than the transverse dimensions of the retainer openings 166 in the patterned frame structures 156a-156e of FIGS. 23-34 to accommodate the longer pairs of extensions 172.

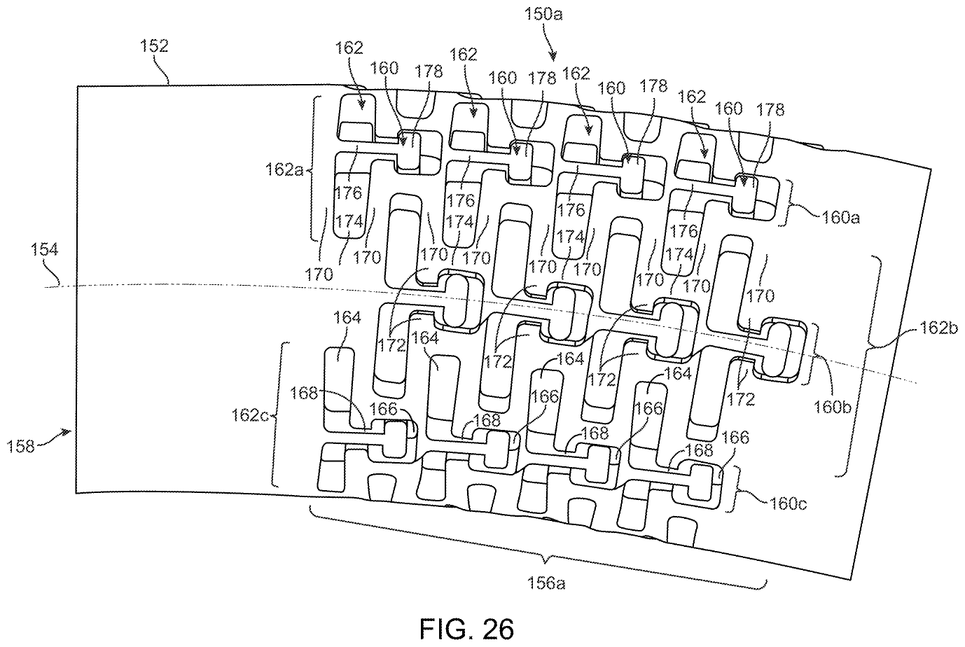

In the illustrated embodiment, only a single one of the floating tab columns is responsible for modulating the bending stiffness of any of the tubular support structures 150a-150f when laterally deflected in a bending direction toward the respective floating tab column (e.g., with respect to the tubular support structure 150a illustrated in FIG. 26, the floating tab column 160b that is on the outer edge of the curve will be active, while the remaining seven floating tab columns 160a and 160c-160h will be inactive). To this end, each retainer opening 166 and associated floating tab 160 of each of the patterned frame structures 156a-156f are arranged, such that there is enough clearance between the floating tab 160 and an edge 186 of respective retainer opening 166 opposite the abutment edge 182 (as illustrated in FIGS. 28A-28B), such that, when the patterned frame structure 156a is laterally deflected in a bending direction within the primary and secondary lateral deflection range that is more than 90 degrees away from the single floating tab column on the outer edge of the curve, the floating tab 160 in each of the other floating tab columns will not engage the edge 186 of the respective retainer opening 166 when axially translated in the axial direction 184b (as best illustrated in FIG. 28A), and will, thus, not contribute to the bending stiffness of the tubular support structures 150a-150f.

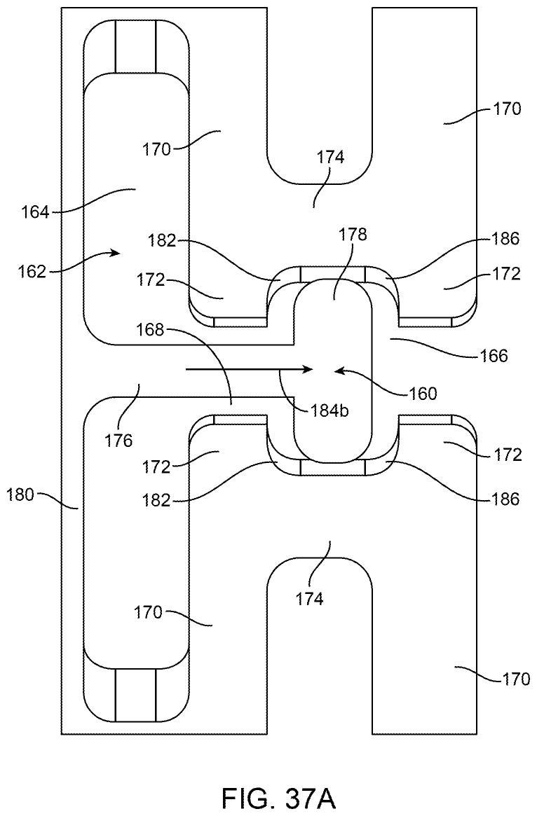

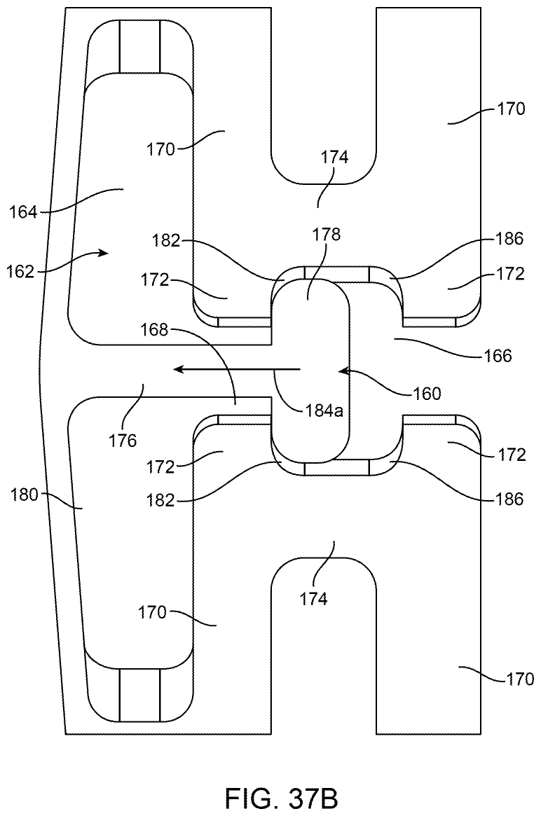

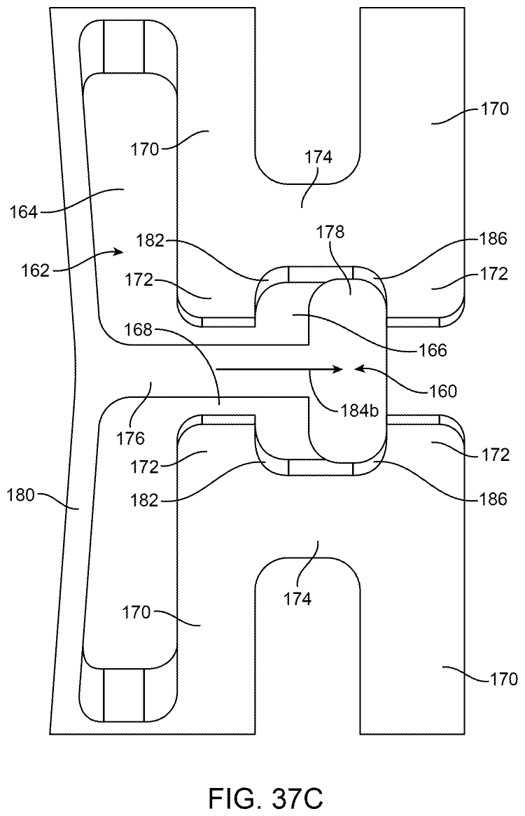

In alternative embodiments, diametrically opposing pairs of floating tab are both responsible for modulating the bending stiffness of any of the tubular support structures 150a-150f when laterally deflected in a bending direction toward one of the diametrically opposing floating tab columns (e.g., with respect to the patterned frame structure 156a illustrated in FIG. 26, the floating tab column 160b that is on the outer edge of the curve and the floating tab column 160f that is on the inner edge of the curve be active, while the remaining six floating tab columns 160a, 160c-160e, and 160g-160h will be inactive), as illustrated in FIGS. 37A-37C.

In this case, it is preferred that two pairs of extensions 172 be provided for each retainer opening 166 to form abutment edges 182, 186 at opposites ends of the retainer opening 166, such that the respective enlarged element 178 may engage either of the abutment edges 182, 186 when axially translated in both directions 184a, 184b, as illustrated in FIGS. 37B-37C. Thus, as illustrated in FIG. 37B, the enlarged element 178 of each floating tab 160 is configured for axially translating in one axial direction 184a within the respective retainer opening 166, thereby engaging the abutment edge 182 of the respective retainer opening 166, when the tubular support structure is laterally deflected in a bending direction toward the floating tab 160 or axially stretched. At this point, the respective floating tab 160 can be considered to be active or activated. Notably, each bridge member 180 to which the stem element 176 of a respective floating tab 160 is affixed flexes to axially translate the enlarged element 178 of the respective floating tab 160 in the axial direction 184a in engagement with the abutment edge 182 of the respective retainer opening 166.

In contrast, as illustrated in FIG. 37A, the enlarged element 178 of each floating tab 160 is configured for axially translating in an opposite axial direction 184b within the respective retainer opening 166, thereby disengaging the abutment edge 182 of the respective retainer opening 166, when the tubular support structure is straightened and/or axially relaxed. At this point, the respective floating tab 160 can be considered to be inactive or deactivated. Notably, each bridge member 180 to which the stem element 176 of a respective floating tab 160 is affixed relaxes to axially translate the enlarged element 178 of the respective floating tab 160 in the axial direction 184b in disengagement with the abutment edge 182 of the respective retainer opening 166.

As illustrated in FIG. 37C, the enlarged element 178 of each floating tab 160 is configured for axially translating further in the direction 184b within the respective retainer opening 166, thereby engaging the abutment edge 186 of the respective retainer opening 166, when the tubular support structure is laterally deflected in a bending direction away from the floating tab 160. At this point, the respective floating tab 160 can be considered to be active or activated. Notably, each bridge member 180 to which the stem element 176 of a respective floating tab 160 is affixed flexes to axially translate the enlarged element 178 of the respective floating tab 160 in the axial direction 184b in engagement with the abutment edge 186 of the respective retainer opening 166.

The retainer openings 166 can be designed, such that at least some of the floating tabs 160 in diametrically opposed floating tab columns engage the respective retainer openings 166 at the same time (to create a secondary bending stiffness) or at different times (to create two secondary bending stiffnesses, a first bending stiffness creating by the floating tabs of one of the diametrically opposed floating tab columns that initially engage the corresponding retainer openings 166, and a secondary bending stiffness creating by the floating tabs of the other diametrically opposed secondary bending stiffness) as the patterned frame structure 156a is laterally deflected in a bending direction toward one of the two opposing floating tab columns.

Notwithstanding the foregoing, it should be appreciated that if any of the tubular support structures 150a-150f is laterally deflected in a bending direction between a pair of adjacent floating tab columns (e.g., the floating tab column 160a and floating tab column 160b of the tubular support structure 150a illustrated in FIG. 26), the respective pair of floating tab columns may contribute to the modulation of the bending stiffnesses of the tubular support structures 150a-150f. In this case, the floating tabs 160 of these adjacent pair of floating tab columns will axially translate in the same direction to engage respective retainer openings 166.

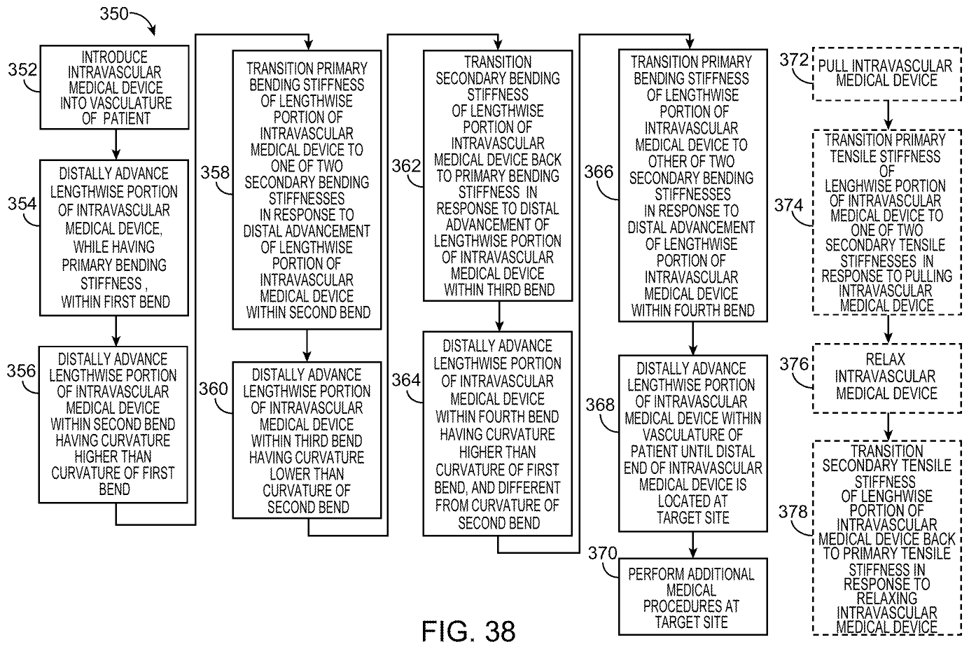

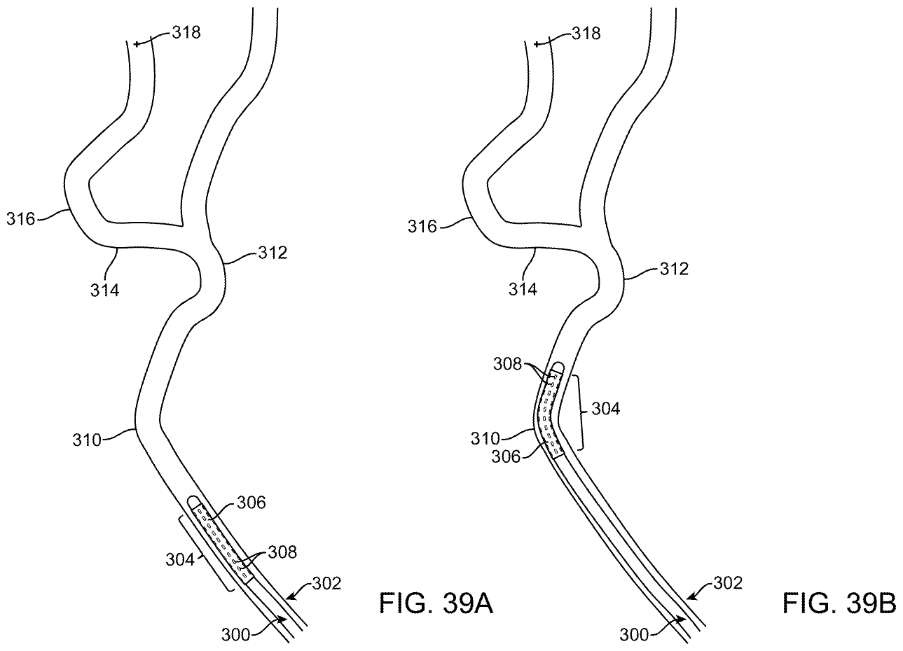

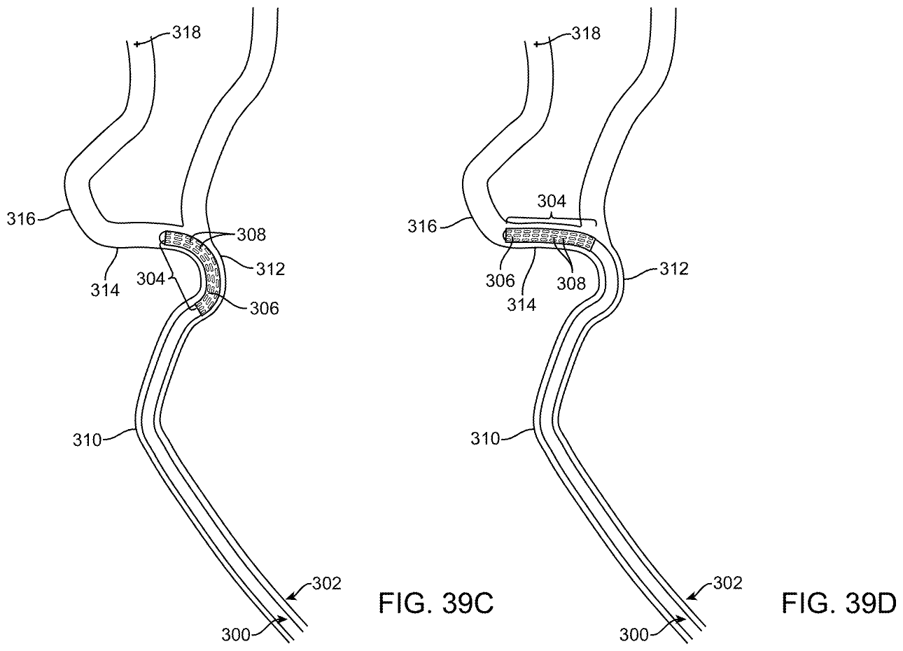

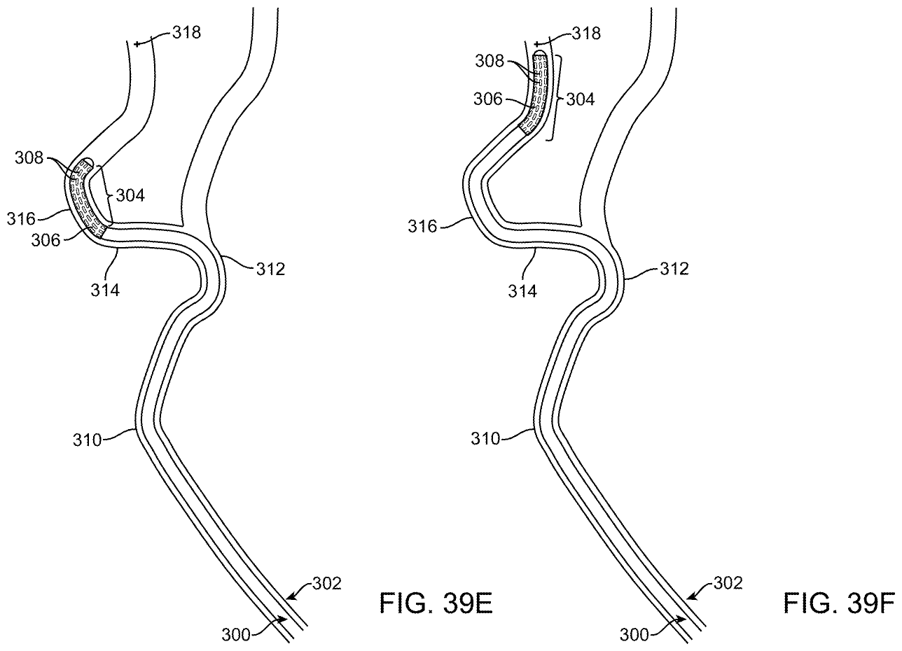

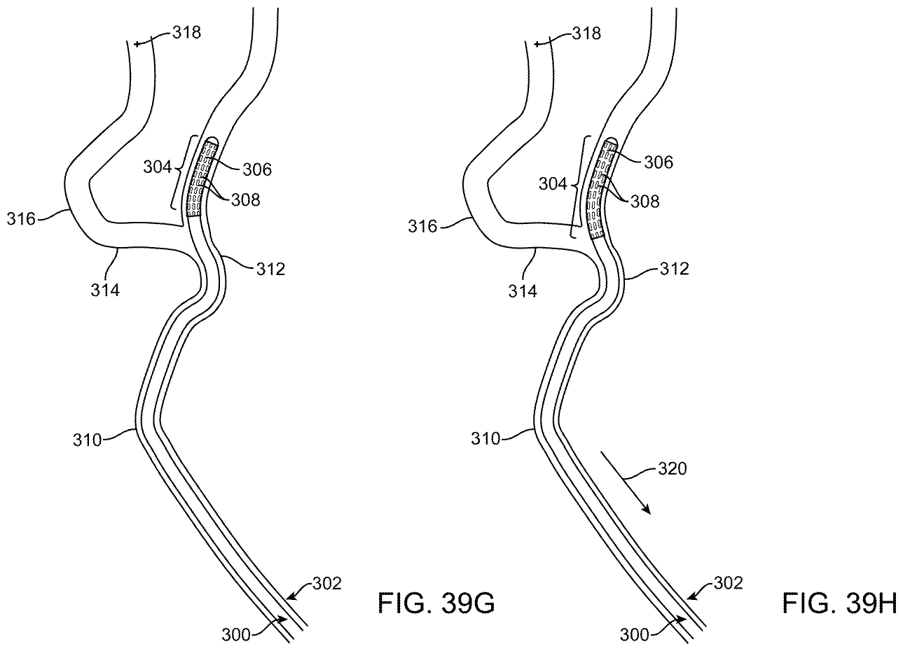

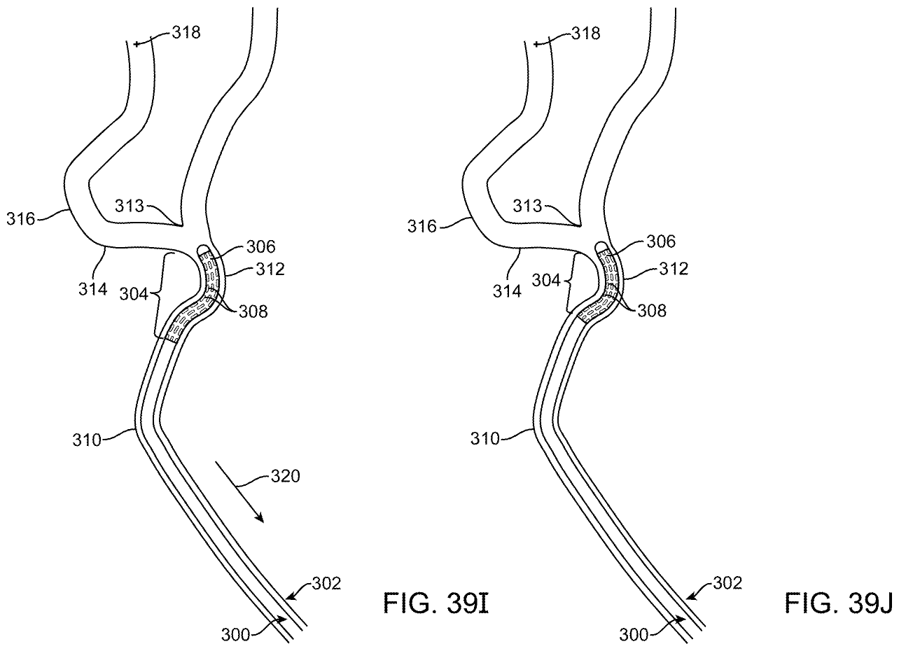

Having described the function and structure of various elongate intravascular medical devices, one method 350 of using an elongate intravascular medical device 300 (e.g., the guidewire 10 illustrated in FIGS. 1-3, the catheter 50 illustrated in FIGS. 4-6, or any other elongated intravascular medical device, such as, e.g., a guide sheath or intravascular implant delivery wire) will now be described with respect to FIGS. 38 and 39A-39H.

In this embodiment, the elongate intravascular medical device 300 has a lengthwise portion 304 containing a tubular support structure 306 (e.g., the tubular support structure 100 illustrated in FIGS. 7-10, or alternatively, the tubular support structure 100′ illustrated in FIG. 11) that is capable of transitioning from a primary bending stiffness to one or more radially isotropic secondary bending stiffnesses (and in this embodiment, one of two secondary bending stiffnesses (e.g., from the primary bending stiffness 200 to one of the lower secondary bending stiffness 202a or the higher secondary bending stiffness 202b illustrated in FIG. 20), and correspondingly is capable of transitioning from a primary tensile stiffness to one or more secondary tensile stiffnesses (and in this embodiment, one of two secondary tensile stiffnesses (e.g., from the primary tensile stiffness 250 to one of the lower secondary tensile stiffness 252a or the higher secondary tensile stiffness 252b illustrated in FIG. 21). In the illustrated embodiment, the lengthwise portion 304 of the elongate intravascular medical device 300 comprises the distal end of the elongate intravascular medical device 300, although in alternative methods, the lengthwise portion 304 of the elongate intravascular medical device 300 may be located proximal to the distal end of the elongate intravascular medical device 300. In the illustrated embodiments, mechanical property modulating elements 308 axially spaced along the tubular support structure 306 are activated to transition the primary bending stiffness to the secondary bending stiffness.

In a conventional manner, the elongate intravascular medical device 300 is first introduced into the vasculature 302 of a patient, e.g., via the femoral artery near the groin of the patient (step 352) (see FIG. 39A).

Next, the lengthwise portion 304 of the elongate intravascular medical device 300 is distally advanced through a first bend 310 in the vasculature 302 of the patient (step 354) (see FIG. 39B). While the lengthwise portion 304 of the elongate intravascular medical device 300 is distally advanced within the first bend 310, the tubular support structure 306, and thus the lengthwise portion 304 of the elongate intravascular medical device 300, has a primary bending stiffness (e.g., the primary bending stiffness 200 illustrated in FIG. 20). That is, the first bend 310 is relatively moderate, such that the tubular support structure 306, and thus the lengthwise portion 304 of the elongate intravascular medical device 300, is laterally deflected within the primary lateral deflection range, and thus, maintains the primary bending stiffness. As such, the lengthwise portion 304 of the elongate intravascular medical device 300 may track through the first bend 310 with relatively little tracking force (due to lower lateral forces).

Next, the lengthwise portion 304 of the elongate intravascular medical device 300 is distally advanced within a second bend 312 having a curvature higher than the curvature of the first bend 310 (step 356) (see FIG. 39C). For the purposes of this specification, a particular bend has a curvature higher than that of another bend in the vasculature of a patient if the minimum radius of curvature of the particular bend is smaller than the minimum radius of curvature of the other bend, and a particular bend has a curvature lower than that of another bend in the vasculature of a patient if the minimum radius of curvature of the particular bend is greater than the minimum radius of curvature of the other bend.

The primary bending stiffness of the tubular support structure 306, and thus the lengthwise portion 304 of the elongate intravascular medical device 300, transitions to one of the two secondary bending stiffnesses in response to the distal advancement of the lengthwise portion 304 of the elongate intravascular medical device 300 within the second bend 312 (e.g., the lower secondary bending stiffness 200a or the higher secondary bending stiffness 200b illustrated in FIG. 20) (step 358). That is, the second bend 312 is relatively high, such that the tubular support structure 306, and thus the lengthwise portion 304 of the elongate intravascular medical device 300, is laterally deflected within the secondary lateral deflection range, and thus, transitions to a secondary bending stiffness. As such, the lengthwise portion 304 of the elongate intravascular medical device 300 will not be prone to prolapse when being introduced through the second bend 312 (due to enhanced support by the tubular support structure 306). Notably, the second bend 312 may not be so high, such that the tubular support structure 306, and thus the lengthwise portion 304 of the elongate intravascular medical device 300, is laterally deflected within the lower region of the secondary lateral deflection range, and thus, transitions to the lower bending stiffness 202a when the lengthwise portion 304 of the elongate intravascular medical device 300 is distally advanced within the second bend 312. Alternatively, the second bend 312 may be high enough, such that the tubular support structure 306, and thus the lengthwise portion 304 of the elongate intravascular medical device 300, is laterally deflected within the upper region of the secondary lateral deflection range, and thus, transitions to the higher bending stiffness 202b when the lengthwise portion 304 of the elongate intravascular medical device 300 is distally advanced within the second bend 312.

Next, the lengthwise portion 304 of the elongate intravascular medical device 300 is distally advanced within a third bend 314 having a curvature less than the curvature of the second bend 312 (step 360) (see FIG. 39D). The secondary bending stiffness of the tubular support structure 306, and thus the lengthwise portion 304 of the elongate intravascular medical device 300, transitions back to the primary bending stiffness (e.g., the primary bending stiffness 202 illustrated in FIG. 20) in response to the distal advancement of the lengthwise portion 304 of the elongate intravascular medical device 300 within the third bend 314 (step 362). That is, the third bend 314 has a low curvature, such that the tubular support structure 306, and thus the lengthwise portion 304 of the elongate intravascular medical device 300, is laterally deflected within the primary lateral deflection range, and thus, transitions back to the primary bending stiffness. As such, the lengthwise portion 304 of the elongate intravascular medical device 300 may track through the third bend 314 with relatively little tracking force (due to lower lateral forces).

Next, the lengthwise portion 304 of the elongate intravascular medical device 300 is distally advanced within a fourth bend 316 having a curvature that is higher than the curvature of the first bend 310, but different from the curvature of the curvature of the second bend 312 (step 364) (see FIG. 39E). The primary bending stiffness of the tubular support structure 306, and thus the lengthwise portion 304 of the elongate intravascular medical device 300, transitions to the other of the two secondary bending stiffnesses in response to the distal advancement of the lengthwise portion 304 of the elongate intravascular medical device 300 within the fourth bend 316 (step 366). That is, the fourth bend 316 is relatively high, such that the tubular support structure 306, and thus the lengthwise portion 304 of the elongate intravascular medical device 300, is laterally deflected within the secondary lateral deflection range, and thus, transitions to a secondary bending stiffness. As such, the lengthwise portion 304 of the elongate intravascular medical device 300 will not be prone to prolapse when being introduced within the fourth bend 316 (due to enhanced support by the tubular support structure 306).

If the curvature of the fourth bend 316 is higher than the curvature of the second bend 312, the secondary bending stiffness to which the primary bending stiffness of the lengthwise portion 304 of the elongate intravascular medical device 300 is transitioned in response to the distal advancement of the lengthwise portion 304 of the elongate intravascular medical device 300 within the fourth bend 316 may be the higher secondary stiffness 202b illustrated in FIG. 20, while the secondary bending stiffness to which the primary bending stiffness of the lengthwise portion 304 of the elongate intravascular medical device 300 is transitioned in response to the distal advancement of the lengthwise portion 304 of the elongate intravascular medical device 300 within the second bend 312 may be the lower secondary stiffness 202a illustrated in FIG. 20.

In contrast, if the curvature of the fourth bend 316 is lower than the curvature of the second bend 312, the secondary bending stiffness to which the primary bending stiffness of the lengthwise portion 304 of the elongate intravascular medical device 300 is transitioned in response to the distal advancement of the lengthwise portion 304 of the elongate intravascular medical device 300 within the fourth bend 316 may be the lower secondary stiffness 202a illustrated in FIG. 20, while the secondary bending stiffness to which the primary bending stiffness of the lengthwise portion 304 of the elongate intravascular medical device 300 is transitioned in response to the distal advancement of the lengthwise portion 304 of the elongate intravascular medical device 300 within the second bend 312 may be the higher secondary stiffness 202b illustrated in FIG. 20.

Of course, in alternative embodiments, the respective curvatures of the second bend 312 and fourth bend 316 may not be so different as result in different secondary bending stiffnesses in response to the distal advancement of the lengthwise portion 304 of the elongate intravascular medical device 300 within the respective second bend 312 or fourth bend 316 in the vasculature 302 of the patient.

The elongate intravascular medical device 300 is distally advanced within the vasculature 302 of the patient until the distal end of the elongate intravascular medical device 300 is located at a target site 318 (step 368) (see FIG. 39F), and additional medical procedures (therapeutic and/or diagnostic) are performed at the target site 318 (step 370). For example, if the elongate intravascular medical device 300 is a guidewire, an additional catheter may be distally advanced over the guidewire to the target site 318 and operated to perform a therapeutic and/or diagnostic procedure at the target site 318. If the elongate intravascular medical device 300 is a catheter, a therapeutic and/or diagnostic procedure may be performed by the catheter at the target site 318. If the elongate intravascular medical device 300 is a guide sheath, an additional catheter may be distally advanced through the guide sheath to the target site 194 and operated to perform a therapeutic and/or diagnostic procedure at the target site 194. If the elongate intravascular medical device 300 is a vascular implant delivery wire, the elongate intravascular medical device 300 may be operated in conjunction with a vascular implant delivery catheter to deploy a vascular implant at the target site 318.

While the elongate intravascular medical device 300 is distally advanced within the vasculature 302 of the patient, the tubular support structure 306, and thus the lengthwise portion 304 of the elongate intravascular medical device 300, has a primary tensile stiffness (e.g., the primary tensile stiffness 250 illustrated in FIG. 21). However, in certain circumstances (e.g., if the distal end of the elongate intravascular medical device 300 has been distally advanced within an undesired portion of the vasculature 302 of the patient (see FIG. 39G) or it is otherwise desired to proximally translate the elongate intravascular medical device 300 to unstick its distal end (e.g., if prolapsed within the vasculature 302 of the patient, becomes trapped by vasospasm or interference with another intravascular medical device, etc.), reorient its distal end, change position relative to a target site, or deploy an intravascular implant from the elongate intravascular medical device 300), the elongate intravascular medical device 300 is pulled (shown by arrow 320) (see FIG. 39H). The primary tensile stiffness of the tubular support structure 306, and thus the lengthwise portion 304 of the elongate intravascular medical device 300, transitions to one of the two secondary tensile stiffnesses (e.g., the lower secondary tensile stiffness 252a or the higher secondary tensile stiffness 252b illustrated in FIG. 20) in response to the pulling of the elongate intravascular medical device 300 (step 374). That is, the elongate intravascular medical device 300 is pulled, such that the tubular support structure 306, and thus the lengthwise portion 304 of the elongate intravascular medical device 300, is axially stretched within the secondary axial stretch range, and thus, transitions to a secondary tensile stiffness. As such, the lengthwise portion 304 of the elongate intravascular medical device 300 will not be prone to plastic deformation or damage. Once the distal end of the elongate intravascular medical device 300 is in a proper location in the vasculature to remedy the problem (e.g., proximal to the bifurcation 313) (see FIG. 39I), the elongate intravascular medical device 300 is then relaxed (step 376) (see FIG. 39J). The secondary tensile stiffness of the tubular support structure 306, and thus the lengthwise portion 304 of the elongate intravascular medical device 300, transitions back to the primary tensile stiffness (e.g., the primary tensile stiffness 250 illustrated in FIG. 20) in response to the relaxation of the elongate intravascular medical device 300 (step 378). The lengthwise portion 304 of the elongate intravascular medical device 300 may then be distally advanced, e.g., within the second bend 312 having a curvature higher than the curvature of the first bend 310 (see FIG. 39C).

It should be appreciated that steps 352-378 may be performed in any order and any number of times, and will highly depend on the nature of the order and number of the bends encountered by the elongate intravascular medical device 300 between the initial introduction of the elongate intravascular medical device 300 into the vasculature 302 of the patient and the time that the distal end of the elongate intravascular medical device 300 is located at the target site 318.

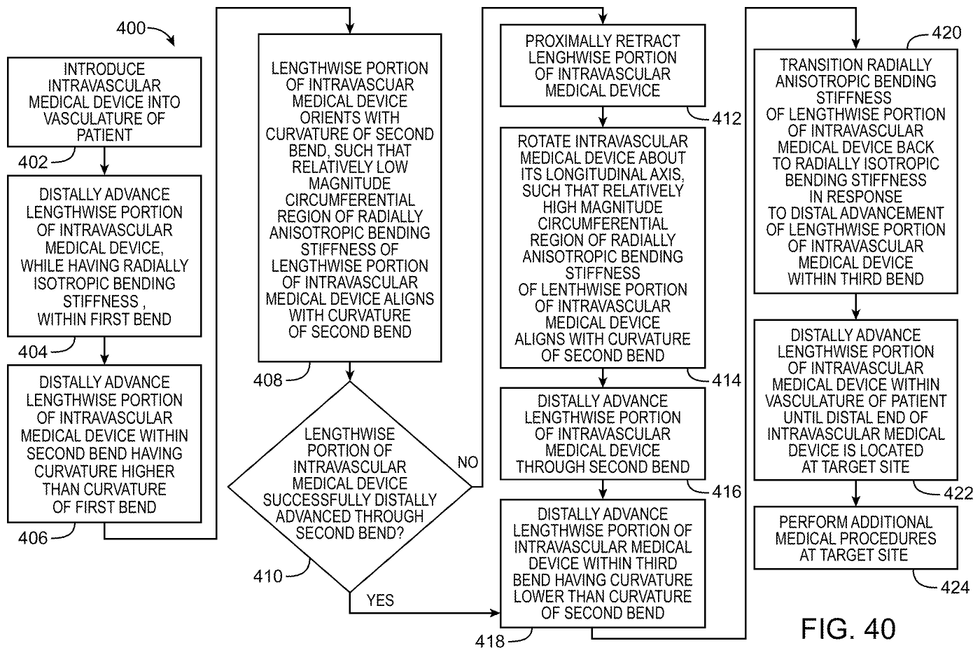

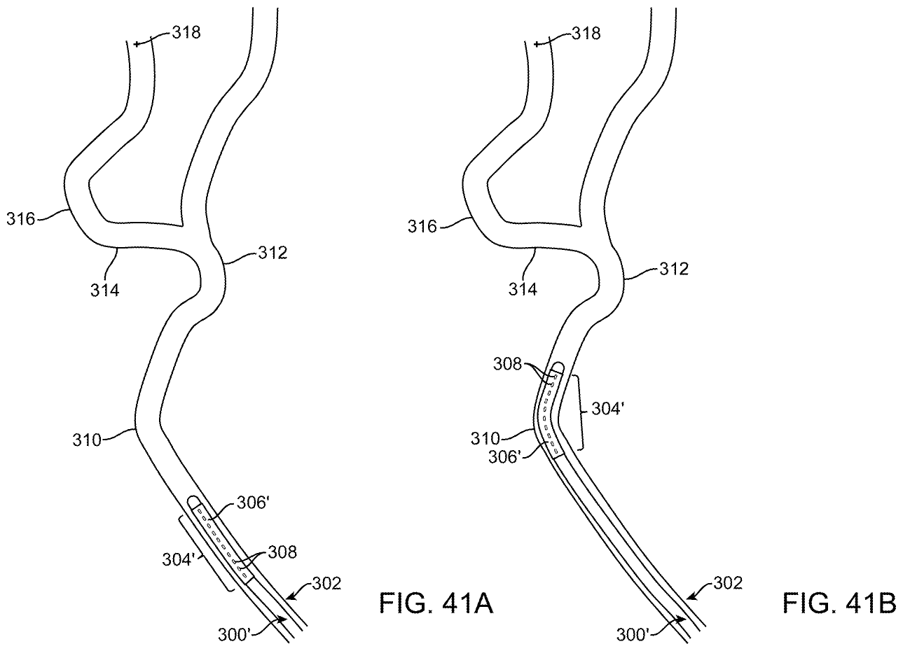

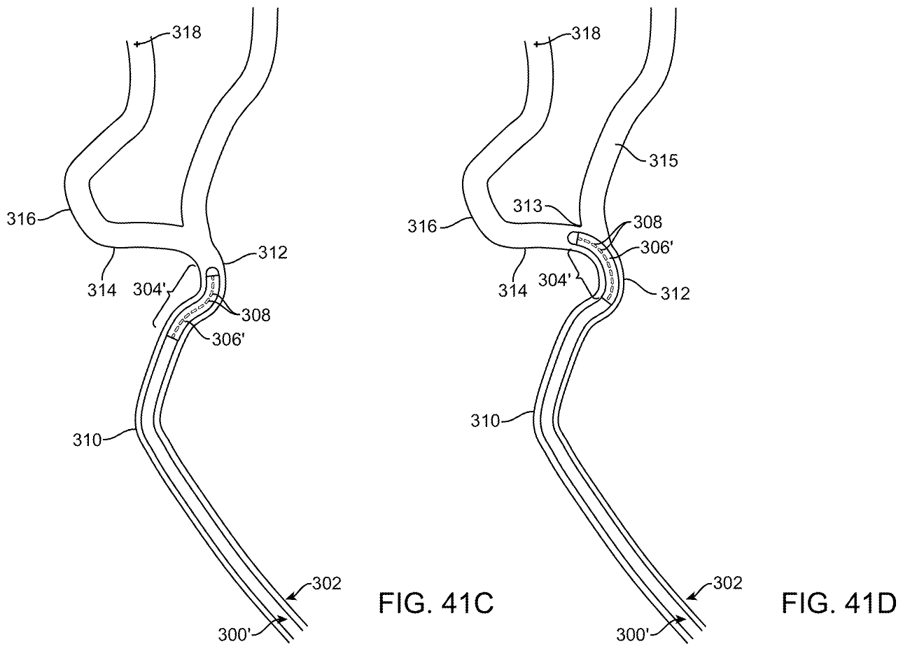

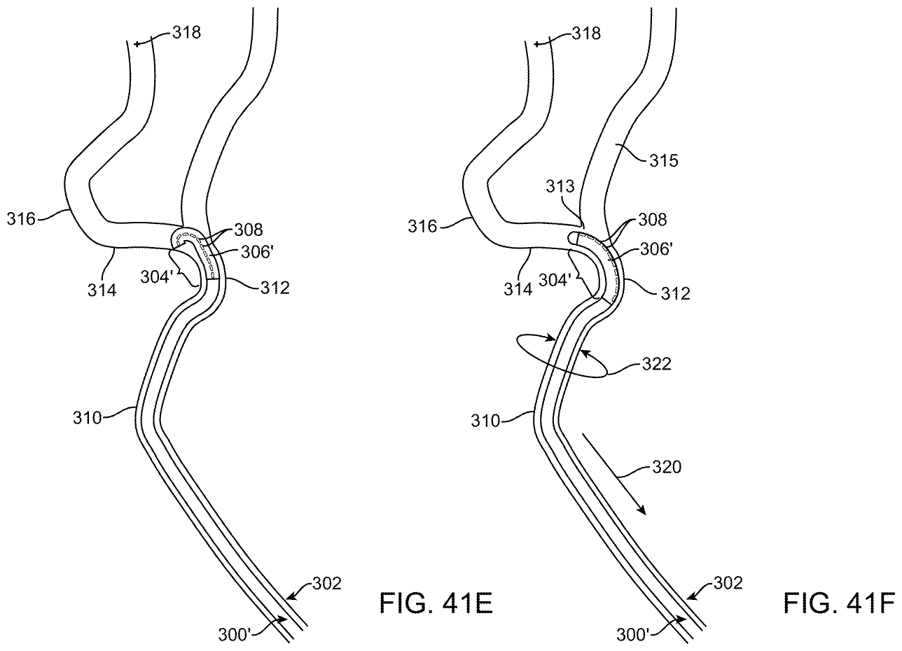

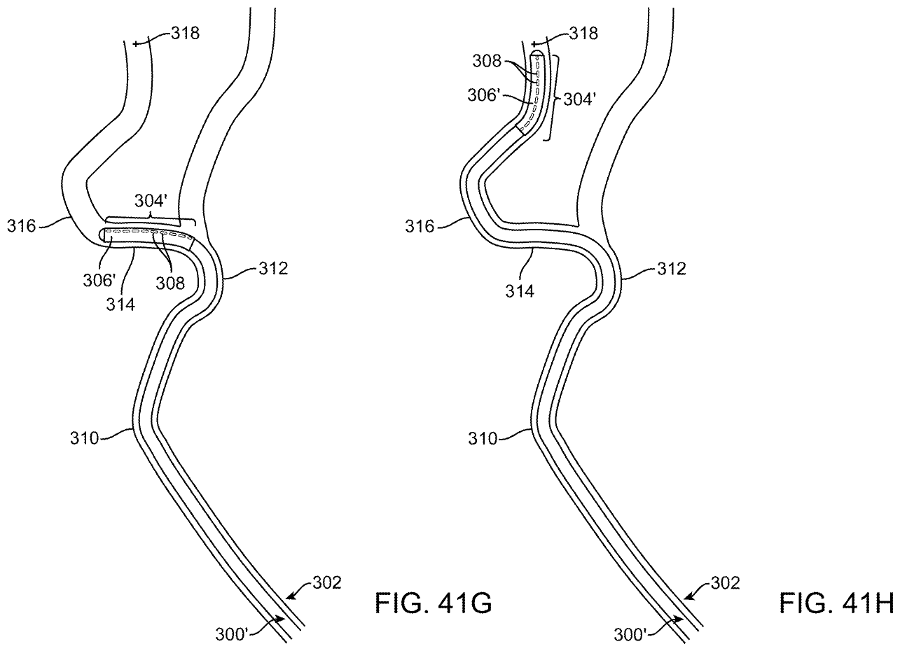

Referring now to FIG. 40 and FIGS. 41A-41I, another method 400 of using an elongate intravascular medical device 300′ (e.g., the guidewire 10 illustrated in FIGS. 1-3, the catheter 50 illustrated in FIGS. 4-6, or any other elongated intravascular medical device, such as, e.g., a guide sheath or intravascular implant delivery wire) will now be described.

In this embodiment, the elongate intravascular medical device 300′ has a lengthwise portion 304′ containing a tubular support structure 306′ (e.g., the tubular support structure 100″ illustrated in FIGS. 12-13, or alternatively, the tubular support structure 100″′ illustrated in FIGS. 15-16) having a radially isotropic bending stiffness (e.g., the primary bending stiffness 200 illustrated in FIG. 20) that is capable of transitioning to a radially anisotropic bending stiffness (e.g., one of the secondary bending stiffnesses 202a, 202b illustrated in FIG. 20) having at least one low magnitude circumferential region 208a and at least one high magnitude circumferential region 208b (e.g., as illustrated in FIGS. 15 and 18-19). In the illustrated embodiment, the lengthwise portion 304′ of the elongate intravascular medical device 300′ comprises the distal end of the elongate intravascular medical device 300′, although in alternative methods, the lengthwise portion 304′ of the elongate intravascular medical device 300′ may be located proximal to the distal end of the elongate intravascular medical device 300′. In the illustrated embodiments, mechanical property modulating elements 308 axially spaced along the tubular support structure 306′ are activated to modulate the bending stiffness of the lengthwise portion 304′ of the elongate intravascular medical device 300′.

The method 400 mainly differs from the method 350 described above with respect to FIG. 38 in that, prior to entering a bend in the vasculature 302 of the patient, the elongate intravascular medical device 300′ may be rotated about its longitudinal axis to select between transitioning the radially isotropic bending stiffness of the lengthwise portion 304′ of the elongate intravascular medical device 300′ to the relatively low magnitude circumferential region 208a or the relatively high magnitude circumferential region 208b of the radially anisotropic bending stiffness when distally advancing the lengthwise portion 304′ of the elongate intravascular medical device 300′ within the bend in the vasculature 302 of the patient.

In the same manner described above with respect to steps 352 and 354, the elongate intravascular medical device 300′ is first introduced into the vasculature 302 of a patient, e.g., via the femoral artery near the groin of the patient (step 402) (see FIG. 41A), and the lengthwise portion 304′ of the elongate intravascular medical device 300′, while having a radially isotropic bending stiffness, is distally advanced within the first bend 310 (step 404) (see FIG. 41B). It is noted that because the curvature of the first bend 310 is not sufficiently high enough to transition the radially isotropic bending stiffness of the lengthwise portion 304′ of the elongate intravascular medical device 300′ to the radially anisotropic bending stiffness, the elongate intravascular medical device 300′ need not be rotated about its longitudinal axis prior to entering the first bend 310 to select between transitioning the radially isotropic bending stiffness of the lengthwise portion 304′ of the elongate intravascular medical device 300′ to the relatively low magnitude circumferential region 208a or the relatively high magnitude circumferential regions 208b of the radially anisotropic bending stiffness.

Next, the lengthwise portion 304′ of the elongate intravascular medical device 300′ is distally advanced within the second bend 312 (step 406) (see FIG. 41C). The curvature of the second bend 312 is sufficiently high enough to transition the radially isotropic bending stiffness of the lengthwise portion 304′ of the elongate intravascular medical device 300′ to a radially anisotropic bending stiffness. Notably, due to the radially anisotropic nature of the secondary bending stiffness, the physician will tend to orient or rotate the lengthwise portion 304′ of the elongate intravascular medical device 300′ with the curvature of the second bend 312, such that the relatively low magnitude circumferential region 208a of the radially anisotropic bending stiffness 202 (shown in FIGS. 15, 18, or 19) of the tubular support structure 306′, and thus the lengthwise portion 304′ of the elongate intravascular medical device 300′ (i.e., the most flexible bending direction of the lengthwise portion 304′ of the elongate intravascular medical device 300′), aligns with the curvature of the second bend 312, so that the distal tip of the elongate intravascular medical device 300′ can continue to be distally advanced along the second bend 312 past the bifurcation 313 instead of being misdirected into the straighter portion 315 of the vasculature 302 past the bifurcation 313 (see FIG. 41D) (step 408). In this manner, the distal tip of the elongate intravascular medical device 300′

If the lengthwise portion 304′ of the elongate intravascular medical device 300′ cannot be successfully distally advanced through the second bend 312 (e.g., if the lengthwise portion 304′ of the elongate intravascular medical device 300′ prolapses within the second bend 312) (see FIG. 41E))(step 410), the distal tip of the elongate intravascular medical device 300′ can be repositioned just beyond the bifurcation in the vasculature 302, e.g., by slightly retracting the lengthwise portion 304′ of the elongate intravascular medical device 300′ (shown by arrow 320) (step 412). The elongate intravascular medical device 300′ may then be rotated about the longitudinal axis (shown by arrow 322), such that the relatively high magnitude circumferential region 208b of the radially anisotropic bending stiffness 202 (shown in FIGS. 15, 18, or 19) of the tubular support structure 306′, and thus the lengthwise portion 304′ of the elongate intravascular medical device 300′ (i.e., the least flexible bending direction of the lengthwise portion 304′ of the elongate intravascular medical device 300′), aligns with the curvature of the second bend 312 (step 414). For example, in the illustrated case, the elongate intravascular medical device 300′ may be rotated about its longitudinal axis until the curvature of the second bend 312 aligns with one of the sets of mechanical property modulating elements 308 (see FIG. 41F). Alternatively, if the tubular support structure 306′ comprises all four of the modulating element columns 110a-110d, with at least two of the modulating element columns 110a-110d modulating the tubular support structure 306′, the elongate intravascular medical device 300′ may be rotated about its longitudinal axis until the curvature of the second bend 312 aligns one of the sets of mechanical property modulating elements 308 corresponding to the high magnitude circumferential region 208b of the radially anisotropic bending stiffness 202. As a result, the radially isotropic bending stiffness of the tubular support structure 306′, and thus the lengthwise portion 304′ of the elongate intravascular medical device 300′, transitions to the relatively high magnitude circumferential region of the radially anisotropic bending stiffness (e.g., one of the secondary bending stiffnesses 202a, 202b illustrated in FIG. 20), and thus, be distally advanced within the second bend 312.

Then, the lengthwise portion 304′ of the elongate intravascular medical device 300′ is distally advanced through the second bend 312 (step 416). Then, in the manner described above with respect to step 360 of FIG. 38, the lengthwise portion 304′ of the elongate intravascular medical device 300′ is distally advanced within the third bend 314 (step 418) (see FIG. 41H), and the radially anisotropic bending stiffness of the tubular support structure 306′, and thus the lengthwise portion 304′ of the elongate intravascular medical device 300′, transitions back to the radially isotropic bending stiffness (e.g., the primary bending stiffness 202 illustrated in FIG. 20) in response to the distal advancement of the lengthwise portion 304′ of the elongate intravascular medical device 300′ within the third bend 314 (step 420). It is noted that because the curvature of the third bend 314 is not sufficiently high enough to transition the radially isotropic bending stiffness of the lengthwise portion 304′ of the elongate intravascular medical device 300′ into the radially anisotropic bending stiffness, the elongate intravascular medical device 300′ need not be rotated about its longitudinal axis prior to entering the third bend 314 to select whether the lengthwise portion 304′ of the elongate intravascular medical device 300′ should be in the relatively low magnitude circumferential region or the relatively high magnitude circumferential region of the radially anisotropic bending stiffness.

Next, the lengthwise portion 304′ of the elongate intravascular medical device 300′ is distally advanced within the fourth bend 316 in the same manner that the elongate intravascular medical device 300′ is distally advanced through the second bend 312 described with respect to steps 406-414. In the same manner described above with respect to steps 368 and 370, the elongate intravascular medical device 300′ is then distally advanced within the vasculature 302 of the patient until the distal end of the elongate intravascular medical device 300′ is located at a target site 318 (step 422) (see FIG. 41I), and additional medical (therapeutic and/or diagnostic) procedures are then performed at the target site 318 (step 424).

It should be appreciated that steps 402-424 may be performed in any order and any number of times, and will highly depend on the nature of the order and number of the bends encountered by the elongate intravascular medical device 300′ between the initial introduction of the elongate intravascular medical device 300′ into the vasculature 302 of the patient and the time that the distal end of the elongate intravascular medical device 300′ is located at the target site 318.

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

1. A tubular support structure for use in an elongate intravascular medical device, comprising:
an elongate tubular body;
a patterned frame structure formed within the elongate tubular body;
an inner lumen axially disposed within the elongate tubular body; and
a first set of floating tabs axially spaced apart along the patterned frame structure, each of the first set of floating tabs having a cantilevered end affixed to the patterned frame structure and a free end configured for translating relative to the patterned frame structure and then engaging the patterned frame structure as the tubular support structure is laterally deflected in a first bending direction.

2. The tubular support structure of claim 1, wherein each of the first set of floating tabs is configured for translating relative to the patterned frame structure when the tubular support structure is in a primary lateral deflection range, and engaging the patterned frame structure when the tubular support structure is in a secondary lateral deflection range greater than the primary lateral deflection range.

3. The tubular support structure of claim 1, wherein the free end of each of the first set of floating tabs is configured for translating relative to the patterned frame structure and then engaging the patterned frame structure as the tubular support structure is axially stretched.

4. The tubular support structure of claim 3, wherein each of the first set of floating tabs is configured for translating relative to the patterned frame structure when the tubular support structure is in a first axial stretch range, and engaging the patterned frame structure when the tubular support structure is in a second axial stretch range greater than the first axial stretch range.

5. The tubular support structure of claim 1, wherein the first set of floating tabs is configured for incrementally engaging the patterned frame structure as the tubular support structure is lateral deflected in the first bending direction.

6. The tubular support structure of claim 5, wherein at least two of the floating tabs have different lengths.

7. The tubular support structure of claim 1, further comprising a second set of floating tabs axially spaced apart along the patterned frame structure and circumferentially offset from the first set of floating tabs, each of the second set of floating tabs having a cantilevered end affixed to the patterned frame structure and a free end configured for translating relative to the patterned frame structure and then engaging the patterned frame structure as the tubular support structure is laterally deflected in a second bending direction different from the first bending direction.

8. The tubular support structure of claim 7, wherein the second set of floating tabs is circumferentially offset from the first set of floating tabs by one hundred eighty degrees, and the second bending direction is opposite the first bending direction.

9. The tubular support structure of claim 7, wherein the first set of floating tabs is configured for translating relative to the patterned frame structure in a first axial direction when the tubular support structure is laterally deflected in the first direction, the second set of floating tabs is configured for translating relative to the frame in a second axial direction opposite the first axial direction.

10. The tubular support structure of claim 9, wherein the first set of floating tabs is configured for continuing to translate in the first axial direction relative to the patterned frame structure after all of the second set of floating tabs have engaged the patterned frame structure, and the second set of floating tabs is configured for continuing to translate in the second axial direction relative to the patterned frame structure after all of the first set of floating tabs have engaged the patterned frame structure.

11. The tubular support structure of claim 1, wherein each of the first set of floating tabs axially extends along the patterned frame structure.

12. The tubular support structure of claim 1, wherein the patterned frame structure has a plurality of substantially transverse slots disposed at least partially within the elongate tubular body, and the substantially transverse slots are axially spaced apart along the tubular support structure, thereby forming a plurality of substantially transverse members and a plurality of connecting members rigidly coupling the substantially transverse members together, wherein the cantilevered end of each of the floating tabs is affixed to a respective one of the substantially transverse members.

13. The tubular support structure of claim 12, wherein the connecting members axially extend along the patterned frame structure.

14. The tubular support structure of claim 12, wherein the substantially transverse slots are disposed entirely through the elongate tubular body.

15. The tubular support structure of claim 1, wherein each of the first set of floating tabs comprises a stem element and an enlarged element that respectively form the cantilevered end and free end of the respective tab.

16. The tubular support structure of claim 15, wherein each of the first set of floating tabs is T-shaped.

17. The tubular support structure of claim 15, wherein the patterned frame structure comprises a plurality of retainer openings disposed at least partially within the elongate tubular body, and the enlarged element of each of the first set of floating tabs is configured for translating within a respective one of the retainer openings and then engaging an abutment edge of the respective retainer opening as the tubular support structure is laterally deflected in a first bending direction.

18. The tubular support structure of claim 17, wherein each of the retainer openings is coextensive with a respective one of the substantially transverse slots, and the stem element of each of the first set of floating tabs extends from the respective transverse member, across a respective one of the substantially transverse slots, and into the respective retainer opening.

19. The tubular support structure of claim 18, wherein each adjacent pair of substantially transverse members comprises a pair of extensions that form a channel between a respective one of the retainer openings and a respective one of the substantially transverse slots that are coextensive with each other, wherein the stem element of each of the first set of floating tabs resides within a respective one of the channels, wherein each pair of extensions defines the abutment edge of the respective retainer opening.

20. The tubular support structure of claim 19, wherein each pair of extensions is configured for laterally flexing when the enlarged element of the respective floating tab engages the abutment edge of the respective retainer opening.

21. An elongate intravascular medical device, comprising:
an elongate polymer tube; and
the tubular support structure of claim 1 coaxially disposed within the polymer tube.

22. The elongate intravascular medical device of claim 21, wherein the tubular support structure is disposed at a distal end of the polymer tube.

23. The elongate intravascular medical device of claim 21, wherein the tubular support structure is disposed proximal to the distal end of the polymer tube.

24. An elongate intravascular medical device, comprising:
a core wire; and
the tubular support structure of claim 1 disposed over a distal end of the core wire.

25. An elongate intravascular medical device, comprising:
tubular support structure of claim 1; and
an inner polymer liner disposed within the inner lumen of the tubular body of the tubular support structure.
